(19) United States
(12) Patent Application Publication
(10) Pub. No.: US 2026/0054982 A1
(43) Pub. Date: Feb. 26, 2026

(54) SYSTEMS AND METHODS FOR PRODUCTION OF LOW CARBON INTENSITY HYDROGEN FROM GEOLOGIC SOURCES

(71) Applicant: Koloma, Inc., Denver, CO (US)

(72) Inventors: David Guro, Coopersburg, PA (US); Erik Scher, San Francisco, CA (US); Peter Johnson, Mountain View, CA (US); Sanford Morton, Boulder, CO (US); Wesley Sellner, Arvada, CO (US)

(21) Appl. No.: 19/309,315

(22) Filed: Aug. 25, 2025

Related U.S. Application Data
(60) Provisional application No. 63/686,596, filed on Aug. 23, 2024.

(51) Int. Cl. C01B 3/56; B01D 53/047; B01D 53/22; C01B 32/40; C01C 1/04; C07C 1/04; C07C 29/151; C07C 29/152; C07C 273/04; C10L 3/08; E21B 21/06; E21B 21/08; E21B 21/16; E21B 43/34

(52) U.S. Cl. CPC C01B 3/56; B01D 53/047; B01D 53/229; C01B 32/40; C01C 1/04; C07C 1/041; C07C 29/1518; C07C 29/152; C07C 273/04; C10L 3/08; E21B 21/062; E21B 21/16; E21B 43/34; B01D 2256/10; B01D 2256/16; B01D 2257/11; B01D 2257/504; B01D 2257/7025; C01B 2203/042; C01B 2203/0475; C01B 2203/061; C01B 2203/062; C01B 2203/068; E21B 21/085

(57) ABSTRACT

A hydrogen production system for producing a hydrogen gas product includes a geologic hydrogen source configured to provide a feedstock comprising hydrogen, nitrogen, and helium and purification equipment comprising two or more of: a pressure swing adsorption (PSA) device; a guard bed; a separation membrane; a reactive membrane; or a cryogenic separation device. The purification equipment is configured to receive the feedstock from the geologic hydrogen source and produce a hydrogen gas product, and production of the hydrogen gas product exhibits a carbon intensity score less than 3.0 kg CO₂ eq/kg H₂.

### CROSS-REFERENCE TO RELATED APPLICATIONS

This application claims benefit to U.S. Provisional Application No. 63/686,596, filed Aug. 23, 2024, the entire disclosure of which is hereby incorporated by reference.

### BACKGROUND

The environmental impact of greenhouse gases (GHGs), primarily carbon dioxide (CO₂) and methane (CH₄), has been the subject of much public debate over the past several decades. More recently, self-imposed private-sector initiatives and government-mandated regulations to reduce the release of greenhouse gases into the environment have begun to be implemented. In addition to the capture and/or sequestration of carbon dioxide and other greenhouse gases to mitigate their atmospheric release, much research and development effort has been focused on the utilization of alternatives to fossil fuels.

Hydrogen (H₂) holds promise as both an energy source and chemical feedstock. However, hydrogen has traditionally been produced using fossil fuels (e.g., via natural gas/methane conversion in a steam methane reformer), and therefore hydrogen has not been viewed as an alternative to the use of fossil fuels. For example, in the steam-methane reforming reaction, methane is reacted with steam (i.e., water) to produce hydrogen gas and carbon monoxide. In a subsequent water-gas shift reaction, the carbon monoxide is further reacted with steam to produce carbon dioxide and additional hydrogen gas. Thus, most hydrogen that is produced in refinery operations, for example, produces greenhouse gases.

Alternatively, hydrogen gas may be generated by the electrolysis of water into hydrogen gas and oxygen. However, producing hydrogen via electrolysis requires a substantial amount of electricity. While at least some of the required electricity for hydrogen production via electrolysis may be obtained from renewable sources (e.g., wind, solar, and hydroelectric), in practice the majority of the electricity used for this purpose has traditionally been, and continues to be, produced through the combustion of fossils fuels, which also produces greenhouse gases.

There is a significant focus today on the decarbonization of energy and chemical industries to positively impact climate change. In response, companies and individuals are actively working to produce cost-effective “clean” or “green” hydrogen and other chemicals. Hydrogen is labelled as “green” when its production results in significantly lower greenhouse gas emissions compared to the production of other energy sources. Governments have recently begun to categorize hydrogen by assessing the emissions intensity of the production plant or system from which the hydrogen is sourced. Specifically, a hydrogen gas product is assigned a carbon intensity (CI) score according to the greenhouse gas emissions resulting from the processing plant.

The production of low CI score hydrogen today is primarily provided through the electrolysis of water, but the need for both renewable electricity and hydrogen gaseous storage, coupled with the high capital cost of the nascent electrolyzer technology, establish that there is a substantial cost to produce this low CI score hydrogen.

### BRIEF SUMMARY

There exists a need for hydrogen production systems and methods that produce low-cost hydrogen gas products having a low CI score that are available without the need for storage.

The disclosure herein provides example embodiments of hydrogen production systems and methods that address this need. As an alternative to producing hydrogen from natural gas via steam-methane reforming or electrolysis, another less-explored option is natural hydrogen produced from subsurface geologic H₂ accumulations. Estimates for the natural H₂ flux from the earth vary widely but tend to be of order-of-magnitude 0.1-10 Tg H₂/year. Over time, the estimates of H₂ flux from the earth have increased, and some estimation methods suggest much larger subsurface production rates and rates of surface hydrogen fluxes are possible. If larger production and flux values are correct, or long-term subsurface accumulation has occurred, natural H₂ could provide a significant amount of low-carbon energy. Natural H₂ has not been commercially developed at scale to date, and wells have only recently been drilled for purposeful production. In order for natural hydrogen to be a useful part of clean energy systems, natural hydrogen will need to be processed in systems and methods with minimal carbon emissions.

The extraction, separation, and purification of geologic hydrogen solves the cost, storage, and scalable volume of supply challenges for hydrogen described above. Hydrogen reserves relate to accumulations stored in subsurface reservoirs contained within the geologic formations underground, and, with proper analysis and planning, hydrogen that can be extracted from boreholes into the subsurface from wellheads is of a sufficient composition that it can be subsequently separated and/or purified to between about 90% and about 99.9999% purity, meeting the needs of the hydrogen markets. With proper equipment selection and potentially clean on-site power generation, the hydrogen is produced with a low CI score.

Further, many of the gas streams extracted from the targeted wellheads contain helium. With creative gas processing through equipment selection and process design as described herein, a helium gas product may also be produced at a low cost and low CI score.

Example embodiments of the present invention set forth herein process feedstock from a geologic hydrogen source to produce a hydrogen gas product with a low CI score. In one or more embodiments, the production of the hydrogen gas product exhibits a carbon intensity score less than 4.0 kg/CO₂ eq/kg H₂, or less than 3.0 kg/CO₂ eq/kg H₂.

Additionally, as outlined below, example embodiments described herein demonstrate the production of low CI score helium, neon, krypton, or xenon in addition to low CI score hydrogen. Finally, some example embodiments described herein produce low CI score ammonia, in which a low CI score hydrogen gas product is used as feedstock to the ammonia loop, while also producing low CI score helium.

An example hydrogen production system for producing a hydrogen gas product includes a geologic hydrogen source and purification equipment comprising one or more of: a pressure swing adsorption (PSA) device; a guard bed; a separation membrane; a reactive membrane; or a cryogenic separation device. The purification equipment is configured to receive feedstock from the geologic hydrogen source and produce a hydrogen gas product, and production of the hydrogen gas product exhibits a carbon intensity score less than 3.0 kg CO₂ eq/kg H₂.

Further, an example method of producing a hydrogen gas product includes: receiving feedstock from a geologic hydrogen source; processing the feedstock using purification equipment; and producing the hydrogen gas product, wherein production of the hydrogen gas product exhibits a carbon intensity score less than 3.0 kg CO₂ eq/kg H₂.

In another embodiment, a hydrogen production system includes a geologic hydrogen source configured to provide a feedstock and purification equipment. The feedstock comprises hydrogen, nitrogen, and helium, among other components, and has a helium molar fraction greater than 0.1 mol %. The feedstock may also comprise neon, krypton, or xenon, and has molar fractions of these additional components greater than 0.05 mol %, greater than 100 parts per million (ppm), or greater than 5 parts per million. The purification equipment includes one or more of: a PSA device; a guard bed; a separation membrane; a reactive membrane; or a cryogenic separation device. The purification equipment is configured to receive the feedstock from the geologic hydrogen source and produce a hydrogen gas product.

In a further embodiment, a method of producing a hydrogen gas product includes: receiving feedstock from a geologic hydrogen source, wherein the feedstock comprises hydrogen, nitrogen, and helium, and wherein the feedstock has a helium molar fraction greater than 0.1 mol %, greater than 0.5 mol %, greater than 1.0 mol %, greater than 2.0 mol %, greater than 3.0 mol %, greater than 4.0 mol %, or greater than 5.0 mol %; processing the feedstock using purification equipment, and producing the hydrogen gas product. The purification equipment includes one or more of: a PSA device; a guard bed; a separation membrane; a reactive membrane; or a cryogenic separation device.

In another embodiment, a hydrogen production system includes a geologic hydrogen source configured to provide a feedstock comprising hydrogen that is not produced using electrolysis, steam methane reformation, methane pyrolysis, or gasification, and purification equipment comprising one or more of: a PSA device; a guard bed; a separation membrane; a reactive membrane; or a cryogenic separation device. The purification equipment is configured to receive the feedstock from the geologic hydrogen source and produce a hydrogen gas product.

In a further embodiment, a method of producing a hydrogen gas product includes: receiving feedstock from a geologic hydrogen source, wherein the feedstock comprises hydrogen that is not produced using electrolysis, steam methane reformation, methane pyrolysis, or gasification; processing the feedstock using purification equipment; and producing the hydrogen gas product. The purification equipment comprises one or more of: a PSA device; a guard bed; a separation membrane; a reactive membrane; or a cryogenic separation device.

In various embodiments, a hydrogen production system includes one or more guard beds configured to receive feedstock from a geologic hydrogen source, wherein each guard bed is configured to remove feedstock impurities such as but not limited to water, carbon dioxide, or hydrocarbons, from the feedstock to produce a guard bed effluent, wherein the guard bed effluent comprises geologic hydrogen; a PSA device configured to receive the guard bed effluent from the one or more guard beds, wherein the PSA device produces a PSA device effluent and a purge gas stream containing unrecovered hydrogen and helium as well as remaining PSA feedgas components including but not limited to water, nitrogen, carbon dioxide, or hydrocarbons, the PSA device effluent comprising hydrogen and helium, and the purge gas stream comprising unrecovered hydrogen and remaining feedstock components comprising nitrogen, carbon dioxide, or methane; a membrane configured to receive the PSA device effluent from the PSA device and to remove nitrogen, carbon dioxide, and/or methane components from the PSA device effluent, wherein the membrane is configured to produce a membrane effluent comprising hydrogen and helium; and a cryogenic separation device configured to receive the membrane effluent from the membrane and to remove helium from the membrane effluent, wherein the cryogenic separation device is configured to produce a hydrogen gas product comprising hydrogen. Said guard beds remove feedstock impurities such as sulfur, particulates, metals, or liquid components from the feedstock.

In further embodiments, a method of producing a hydrogen gas product includes: receiving, by one or more guard beds, feedstock from a geologic hydrogen source; removing, by the one or more guard beds, feedstock impurities from the feedstock; receiving, by a PSA device, a guard bed effluent from the one or more guard beds, wherein the guard bed effluent comprises geologic hydrogen; producing, by the PSA device, a PSA device effluent and a purge gas stream, the PSA device effluent comprising hydrogen and helium, and the purge gas stream comprising unrecovered hydrogen and remaining feedstock components comprising nitrogen, carbon dioxide, or methane; receiving, by a membrane, the PSA device effluent from the PSA device; removing, by the membrane, nitrogen, carbon dioxide, and/or methane components from the PSA device effluent; producing, by the membrane, a membrane effluent comprising hydrogen and helium; receiving, by a cryogenic separation device, the membrane effluent from the membrane; removing, by the cryogenic separation device, helium from the membrane effluent; and producing, by the cryogenic separation device, a hydrogen gas product comprising hydrogen.

In other embodiments, a hydrogen production system includes one or more guard beds configured to receive feedstock from a geologic hydrogen source, each guard bed configured to remove feedstock impurities from the feedstock, wherein the guard bed effluent comprises geologic hydrogen; a PSA device configured to receive guard bed effluent from the one or more guard beds and produce a PSA device effluent and a purge gas stream, wherein the PSA device effluent comprises hydrogen and helium, and wherein the purge gas stream comprises unrecovered hydrogen and remaining feedstock components comprising nitrogen or carbon dioxide or methane; and a reactive membrane configured to receive the PSA device effluent and remove hydrogen from the PSA device effluent, wherein the reactive membrane is configured to produce a first gas stream comprising predominantly hydrogen and a second gas stream comprising helium.

In certain embodiments, the purge gas produced by the hydrogen or first-stage purification equipment described herein comprises carbon dioxide in admixture with unrecovered hydrogen, nitrogen, methane and/or helium. Optionally, at least a portion of this carbon dioxide-containing purge gas or off gas is separated from hydrogen by a carbon dioxide purification equipment including one or more of: (i) a secondary PSA; (ii) temperature swing or vacuum swing adsorption; (iii) physical or chemical solvent absorption (e.g., amine, carbonate or caustic scrubbing); (iv) membrane separation; or (v) cryogenic distillation. The resulting carbon dioxide-rich gas stream, typically having a carbon dioxide molar fraction of at least 50 mol %, more preferably greater than 90 mol %, may be routed to an offtake facility that converts carbon dioxide, together with at least a portion of the hydrogen gas product or hydrogen produced by the hydrogen purification equipment, to one or more value-added products including, without limitation, synthetic natural gas, methanol, ethanol, Fischer-Tropsch hydrocarbons (e.g., sustainable aviation fuel), methanol to gasoline (MTG) or methane to olefin (MTO) products, carbon monoxide via reverse water gas shift (RWGS), or carbamide products such as urea or urea ammonium nitrate (UAN) fertilizer. Optionally, the hydrogen and carbon dioxide streams produced topside may be delivered directly, without intermediate storage, to an integrated offtake facility, such as an RWGS or Fischer-Tropsch reactor located near the hydrogen purification facility, thereby minimizing additional carbon contributions from compression or trucking.

In further embodiments, a method includes: receiving, by one or more guard beds, feedstock from a geologic hydrogen source; removing, by the one or more guard beds, drier beds, or knock-out vessels, feedstock impurities from the feedstock; receiving, by a PSA device, a guard bed effluent from the one or more guard beds, wherein the guard bed effluent comprises geologic hydrogen; producing, by the PSA device, a PSA device effluent and a purge gas stream, the PSA device effluent comprising hydrogen and helium, and the purge gas stream comprising unrecovered hydrogen and remaining feedstock components comprising nitrogen or carbon dioxide or methane; receiving, by a reactive membrane, the PSA device effluent from the PSA device; removing, by the reactive membrane, hydrogen from the PSA device effluent; and producing, by the reactive membrane, a first gas stream comprising predominantly hydrogen and a second gas stream comprising helium.

In some embodiments, a hydrogen production system includes a wellhead configured to provide a feedstock including hydrogen and purification equipment. The purification equipment includes one or more of: a PSA device; a guard bed; a separation membrane; a reactive membrane; or a cryogenic separation device. The purification equipment is configured to receive the hydrogen feedstock from the wellhead and produce a hydrogen gas product.

In further embodiments, a method of producing a hydrogen gas product includes: receiving hydrogen feedstock from a hydrogen source, wherein the hydrogen source is a wellhead; processing the hydrogen feedstock using purification equipment; and producing the hydrogen gas product. The purification equipment comprising one or more of: a PSA device; a guard bed; a separation membrane; a reactive membrane; or a cryogenic separation device.

In other embodiments, a hydrogen production system includes a geologic hydrogen source configured to provide a feedstock, purification equipment configured to receive the feedstock from the geologic hydrogen source and produce a hydrogen gas product, and a power generation plant powered by hydrogen from one of the feedstock, the purification equipment, or purge gas from the purification equipment. The power generation plant is configured to provide energy to the purification equipment.

In still further embodiments, a method of producing a hydrogen gas product includes: receiving feedstock from a geologic hydrogen source; processing the feedstock using purification equipment; producing, by the purification equipment, the hydrogen gas product; receiving, by a power generation plant, hydrogen from one of the feedstock, the purification equipment, or purge gas from the purification equipment; and providing, by the power generation plant, energy to the purification equipment.

In some embodiments, a hydrogen production system includes a geologic hydrogen source, first-stage purification equipment, and second-stage purification equipment. The first-stage purification equipment includes one or more of: a PSA device; a guard bed; a separation membrane; a reactive membrane; or a cryogenic separation device. The first-stage purification equipment is configured to receive feedstock from the geologic hydrogen source and produce an effluent. The second-stage purification equipment configured to receive the effluent from the first-stage purification equipment and produce a hydrogen gas product and a noble gas product.

In other embodiments, a method of producing a hydrogen gas product includes: receiving feedstock from a geologic hydrogen source; processing, by first-stage purification equipment, the feedstock; producing, by the first-stage purification equipment, an effluent; processing, by second-stage purification equipment, the effluent from the first-stage purification equipment; and producing, by the second-stage purification equipment, a noble gas product and the hydrogen gas product.

In some embodiments, a hydrogen production system includes a hydrogen source configured to provide a feedstock, first-stage purification equipment, an ammonia synthesis loop, and second-stage purification equipment. The first-stage purification equipment includes one or more of: a PSA device; a guard bed; a separation membrane; a reactive membrane; or a cryogenic separation device. The first-stage purification equipment is configured to receive the feedstock from the hydrogen source and produce a hydrogen gas product, wherein the hydrogen gas product comprises hydrogen and helium. The ammonia synthesis loop is configured to receive the hydrogen gas product from the first-stage purification equipment and produce an ammonia product and an effluent. The second-stage purification equipment is configured to receive the effluent from the ammonia synthesis loop and produce a helium gas product.

In other embodiments, a method of producing a hydrogen gas product includes: receiving feedstock from a hydrogen source; processing, by first-stage purification equipment, the feedstock; producing, by the first-stage purification equipment, the hydrogen gas product; receiving, by an ammonia synthesis loop, the hydrogen gas product from the first-stage purification equipment; producing, by the ammonia synthesis loop, an ammonia product and an effluent; receiving, by a second-stage purification equipment, the effluent from the ammonia synthesis loop; and producing, by the second-stage purification equipment, a helium gas product. Said helium gas product may comprise other noble gases.

In some example embodiments, a hydrogen production system includes a geologic hydrogen source configured to provide a feedstock and purification equipment configured to receive the feedstock from the geologic hydrogen source and produce a hydrogen gas product. The hydrogen gas product comprises hydrogen, helium, nitrogen, carbon monoxide, carbon dioxide, and methane. A combined hydrogen and helium molar fraction is greater than 95 mol %. For example, the combined hydrogen and helium molar fraction may be greater than 96%, greater than 96%, greater than 97%, or greater than 98%. A nitrogen molar fraction is less than 2 mol %. A combined carbon monoxide and carbon dioxide concentration is less than 50 ppm, and a methane concentration is less than 50 ppm.

In some embodiments, a method of producing a hydrogen gas product includes receiving feedstock from a geologic hydrogen source; processing the feedstock using purification equipment; producing, by the purification equipment, the hydrogen gas product. The hydrogen gas product comprises: a combined hydrogen and helium molar fraction of greater than 98 mol %; a nitrogen molar fraction of less than 1 mol %; a combined carbon monoxide and carbon dioxide concentration of less than 20 ppm; and a methane concentration of less than 20 ppm.

In other embodiments, a hydrogen production system includes a geologic hydrogen source, a compressor receiving feedstock from the geologic hydrogen source, purification equipment, and/or a power generation plant. The purification equipment comprising one or more of: a PSA device; a guard bed; a separation membrane; a reactive membrane; or a cryogenic separation device. The power generation plant is powered by purge gas from the purification equipment and configured to provide energy to one or more of the compressor or the purification equipment. The purification equipment is configured to receive the feedstock and produce a hydrogen gas product.

In still further embodiments, a method of producing a hydrogen gas product includes: receiving feedstock from a geologic hydrogen source; pressurizing the feedstock using a compressor; processing the feedstock using purification equipment to produce the hydrogen gas product; powering a power generation plant using purge gas from the purification equipment; and directing energy from the power generation plant to one or more of the compressor or the purification equipment.

One or more embodiments of this disclosure generally relate to hydrogen production systems and methods that receive feedstock from a geologic hydrogen source, process the hydrogen feedstock to produce a hydrogen gas product. In one or more embodiments, the production of the hydrogen gas product exhibits a carbon intensity score less than 3.0 kg/CO₂ eq/kg H₂.

In some embodiments, the geologic source is accessed through a wellhead. In various embodiments, the hydrogen feedstock includes hydrogen, nitrogen, and helium and has a helium molar fraction greater than 0.1 mol %. In various embodiments, the hydrogen feedstock comprises hydrogen that is not produced through electrolysis, steam methane reformation, methane pyrolysis, or gasification.

In some embodiments, the hydrogen production system includes a power generation plant that is powered by hydrogen from the hydrogen feedstock or the purification equipment. In some embodiments, the hydrogen production system includes a first-stage purification equipment to produce hydrogen and a second-stage purification equipment to produce one or more noble gas products. In some embodiments, the hydrogen production system includes an ammonia synthesis loop configured to receive the hydrogen gas product and to produce an effluent.

In some embodiments, the hydrogen gas product includes hydrogen, helium, and nitrogen, wherein a hydrogen and helium molar fraction is greater than 98% and a nitrogen molar fraction is less than 2%. In some embodiments, the hydrogen gas product includes less than 50 ppm carbon monoxide and carbon dioxide in combination and/or less than 50 ppm methane.

In some embodiments, a method of producing a hydrogen gas product and a carbon dioxide-rich gas stream is presented. The method includes receiving feedstock from a geologic hydrogen source; separating, through a first pressure swing adsorption (PSA) device in a hydrogen purification equipment, carbon dioxide from hydrogen in the feedstock to provide an effluent including hydrogen and a first purge gas including carbon dioxide; separating, through carbon dioxide purification equipment, carbon dioxide from other gases in the first purge gas to provide a carbon dioxide-rich gas stream having a carbon dioxide molar fraction of at least 50 mol % and a second purge gas; and reacting, in a reactor, the carbon dioxide-rich gas stream with hydrogen of the effluent to produce a synthesis product, wherein the synthesis product includes one or more of synthetic natural gas, carbon monoxide, methanol, ethanol, Fischer-Tropsch hydrocarbons, olefins, urea, ammonia, or urea ammonium nitrate.

In some embodiments, the reacting step includes reacting the carbon dioxide-rich stream and the hydrogen in a reverse water gas shift reactor to produce carbon monoxide; and reacting the carbon monoxide with hydrogen in a Fischer-Tropsch reactor. In some embodiments, a combined carbon dioxide conversion is at least 60%.

In some embodiments, the reactor is a batch reactor or a continuous flow reactor.

In some embodiments, the reactor is a bioreactor, and the synthesis product includes ethanol or methane. In some embodiments, the reactor is a reverse water gas shift reactor, and the synthesis product includes carbon monoxide. In some embodiments, the reactor is a Fischer-Tropsch reactor, and the synthesis product includes hydrocarbons. In some embodiments, the reactor is a urea reactor, and the synthesis product is urea. In some embodiments, the reactor is a tubular reactor, and wherein the synthesis product is methanol. In some embodiments, the reactor is a methanation reactor, and wherein the synthesis product is synthetic natural gas.

Some embodiments further include separating, through a third PSA device in the hydrogen purification equipment, nitrogen from hydrogen in the feedstock or the effluent from the first PSA device to provide a nitrogen-rich gas stream; and directing the nitrogen-rich gas stream to an ammonia plant.

In some embodiments, the method includes powering a power generation plant fluidically coupled to the hydrogen purification equipment using the first purge gas of the first PSA device and/or the second purge gas of the carbon dioxide purification equipment.

In some embodiments, the hydrogen purification equipment is configured to receive the feedstock and produce the hydrogen gas product, and wherein production of the hydrogen gas product exhibits a carbon intensity score less than 4.0 kg CO₂ eq/kg H₂.

In some embodiments, production of the hydrogen gas product exhibits a carbon intensity score less than 0.45 kg CO₂ eq/kg H₂.

In some embodiments, the carbon dioxide purification equipment is selected from a second PSA device, a temperature-swing adsorption unit, a separation membrane, a chemical-solvent absorption unit, or a cryogenic distillation unit. In some embodiments, the carbon dioxide-rich gas stream has a carbon dioxide molar fraction of at least 90 mol %.

In some embodiments, the reactor and the hydrogen purification equipment are co-located and the carbon dioxide-rich stream is delivered without intermediate storage.

Some embodiments include a system for producing a hydrogen gas product and a carbon dioxide-rich gas stream. The system includes hydrogen purification equipment configured to receive feedstock from a geologic hydrogen source, wherein the hydrogen purification equipment is configured for separating carbon dioxide from hydrogen in the feedstock to provide an effluent including hydrogen and a first purge gas including carbon dioxide, wherein the hydrogen purification equipment includes a first pressure swing adsorption (PSA) device; carbon dioxide purification equipment for separating carbon dioxide from other gases in the first purge gas to provide the carbon dioxide-rich gas stream having a carbon dioxide molar fraction of at least 50 mol % and a second purge gas, wherein the carbon dioxide purification equipment includes a secondary PSA device, a temperature-swing adsorption, a separation membrane, a chemical-solvent absorption, or a cryogenic distillation; and a reactor configured to receive hydrogen of the effluent and the carbon dioxide-rich gas stream and to produce a synthesis product, wherein the synthesis product includes one or more of synthetic natural gas, carbon monoxide, methanol, ethanol, Fischer-Tropsch hydrocarbons, olefins, urea, and urea ammonium nitrate.

In some embodiments, the system further includes an offtake facility including the reactor.

In some embodiments, the offtake facility includes a reverse water gas shift reactor and a Fischer-Tropsch reactor, and wherein the offtake facility is configured to produce the synthesis product with a combined carbon dioxide conversion of at least 60%.

In some embodiments, the reactor is a batch reactor or a continuous flow reactor.

In some embodiments, the reactor is a bioreactor, and the synthesis product includes ethanol or methane. In some embodiments, the reactor is a reverse water gas shift reactor and the synthesis product includes carbon monoxide. In some embodiments, the reactor is a reverse water gas shift reactor, and wherein the synthesis product includes carbon monoxide. In some embodiments, the reactor is a Fischer-Tropsch reactor, and wherein the synthesis product includes alkanes. In some embodiments, the reactor is a urea reactor, and wherein the synthesis product is urea. In some embodiments, the reactor is a tubular reactor, and wherein the synthesis product is methanol. In some embodiments, the reactor is a methanation reactor, and wherein the synthesis product is synthetic natural gas.

In some embodiments, the hydrogen purification equipment includes a third PSA device configured to provide a nitrogen-rich gas stream.

In some embodiments, the system further includes a power generation plant fluidically coupled to the hydrogen purification equipment and powered by the purge gas of the first PSA device and/or the second purge gas of the carbon dioxide purification equipment.

In some embodiments, the hydrogen purification equipment is configured to receive the feedstock and produce the hydrogen gas product, and wherein production of the hydrogen gas product exhibits a carbon intensity score less than 4.0 kg CO₂ eq/kg H₂. In some embodiments, production of the hydrogen gas product exhibits a carbon intensity score less than 0.45 kg CO₂ eq/kg H₂.

In some embodiments, the carbon dioxide purification equipment is selected from a second PSA device, a temperature-swing adsorption unit, a separation membrane, a chemical-solvent absorption unit, or a cryogenic distillation unit.

In some embodiments, the carbon dioxide-rich gas stream has a carbon dioxide molar fraction of at least 90 mol %. In some embodiments, the reactor and the hydrogen purification equipment are co-located and the carbon dioxide-rich stream is delivered without intermediate storage.

In some embodiments, a method for providing gas for drilling a borehole is included. The method includes receiving, from a geological hydrogen source, a feedstock including hydrogen, nitrogen, carbon dioxide, methane, neon, argon, and helium; separating the feedstock using hydrogen purification equipment to yield an effluent including the hydrogen and a purge gas having a nitrogen molar fraction of at least 50 mol %; compressing the purge gas to a pressure of at least 1 MPa; and utilizing the compressed purge gas in a drilling fluid in the borehole, wherein the drilling fluid includes an oxygen molar fraction of up to 1 mol %.

In some embodiments, the method further includes blending the compressed purge gas with a nitrogen gas stream provided by a vacuum PSA device to yield a mixture, wherein the mixture has a nitrogen molar fraction of at least 85 mol %. In some embodiments, the mixture is supplied to the drilling fluid.

In some embodiments, the method further includes powering a power generation plant using the feedstock or the purge gas from the hydrogen purification equipment, wherein the power generation plant provides a power purge gas including nitrogen; and blending the compressed purge gas with the power purge gas to yield a mixture, wherein the mixture has a nitrogen molar fraction of at least 85 mol %.

In some embodiments, the mixture is supplied to the drilling fluid. In some embodiments, the purge gas is compressed to at least 1 MPa and has an oxygen molar fraction less than or equal to 1 mol %. In some embodiments, after blending, a nitrogen molar fraction is at least 90 mol %. In some embodiments, the purge gas is supplied directly to a rotating-head underbalanced drilling system.

In some embodiments, the hydrogen purification equipment is configured to receive the feedstock and produce a hydrogen gas product, and wherein production of the hydrogen gas product exhibits a carbon intensity score less than 4.0 kg CO₂ eq/kg H₂.

In some embodiments, a system for supplying drilling fluid is included. The system includes hydrogen purification equipment configured to receive feedstock including hydrogen, nitrogen, carbon dioxide, methane, neon, argon, and helium from a geologic hydrogen source, wherein the hydrogen purification is configured for separating nitrogen from hydrogen in the feedstock to provide an effluent including hydrogen and a purge gas including a nitrogen molar fraction of at least 50 mol %; a compressor configured to pressurize the purge gas to at least 1 MPa; and a discharge line configured to supply the drilling fluid to a drilling rig, wherein the drilling fluid includes an oxygen molar fraction of up to 1 mol %.

In some embodiments, the system further includes a vacuum PSA device configured to produce a nitrogen gas stream from air, wherein the nitrogen gas stream is blended with the pressurized purge gas to yield a mixture having a nitrogen molar fraction of at least 85 mol %. In some embodiments, the mixture is supplied to the drilling fluid.

In some embodiments, the system further includes a power generation plant configured to produce a nitrogen purge gas, wherein the nitrogen purge gas is blended with the pressurized purge gas to yield a mixture, wherein the mixture has a nitrogen molar fraction of at least 85 mol %. In some embodiments, the mixture is supplied to the drilling fluid. In some embodiments, the purge gas is compressed to at least 1 MPa and has an oxygen molar fraction less than or equal to 1 mol %. In some embodiments, after blending, a nitrogen molar fraction is at least 90 mol %. In some embodiments, the purge gas is supplied directly to a rotating-head underbalanced drilling system.

In some embodiments, the hydrogen purification equipment is configured to receive the feedstock and produce a hydrogen gas product, and wherein production of the hydrogen gas product exhibits a carbon intensity score less than 4.0 kg CO₂ eq/kg H₂.

In some embodiments, a system includes a pressure swing adsorption (PSA) device configured to receive feedstock from a geologic hydrogen source and produce a PSA device effluent and a purge gas stream, wherein the PSA device effluent includes hydrogen and helium, and wherein the purge gas stream includes unrecovered hydrogen and remaining feedstock components including nitrogen or carbon dioxide or methane; and a reactive membrane configured to directly or indirectly receive the PSA device effluent and remove hydrogen from the PSA device effluent, wherein the reactive membrane is configured to produce a first gas stream including predominantly hydrogen and a second gas stream including predominantly helium, wherein the reactive membrane includes a metal selected from the group consisting of palladium, a palladium alloy, platinum, rhodium, ruthenium, copper, nickel, silver, titanium, or another metal; wherein the PSA device and the reactive membrane are fluidically coupled to each other.

In some embodiments, the system further includes a compressor fluidically coupled to the geologic hydrogen source.

In some embodiments, the PSA includes one or more layers of one or more adsorbent materials from the list of silica gel, alumina, various zeolites, or activated carbons. In some embodiments, the first gas stream has a hydrogen molar fraction of at least about 90 mol %. In some embodiments, the hydrogen molar fraction is at least about 95 mol %. In some embodiments, the hydrogen molar fraction is at least about 97 mol %. In some embodiments, the hydrogen molar fraction is at least about 99 mol %.

In some embodiments, the second gas stream has a helium molar fraction of at least about 90 mol %. In some embodiments, the helium molar fraction is at least about 95 mol %. In some embodiments, the helium molar fraction is at least about 97 mol %. In some embodiments, the helium molar fraction is at least about 99 mol %.

In some embodiments, the first gas stream has a hydrogen molar fraction of at least about 90 mol %, and wherein the second gas stream has a helium molar fraction of at least about 90 mo1%.

In some embodiments, the system further includes a power generation plant fluidically coupled to the geologic hydrogen source and to the PSA device, wherein the power generation plant is powered by the purge gas stream from the PSA device or the feedstock from the geologic hydrogen source, wherein the power generation plant is configured to provide energy to one or more of a compressor, the PSA device, or the reactive membrane.

In some embodiments, the power generation plant is powered by the feedstock from the geologic hydrogen source only. In some embodiments, excess power that is not consumed by the compressor, the PSA device, or the reactive membrane is directed to an independent system.

In some embodiments, the reactive membrane is positioned downstream of the PSA device and configured to receive a PSA effluent including hydrogen and helium. In some embodiments, the reactive membrane is configured to produce (a) a helium-enriched permeate having a helium molar fraction greater than 90 mol %, and (b) a hydrogen-enriched retentate having a hydrogen molar fraction greater than 95 mol %. In some embodiments, the PSA device effluent of the PSA device includes hydrogen and helium.

In some embodiments, the system further includes a separation device configured to receive the PSA device effluent from the PSA device and produce a separation device effluent and a purge gas stream, wherein the separation device effluent includes predominantly hydrogen and helium, and wherein the reactive membrane is configured to receive the separation device effluent.

In some embodiments, production of the first gas stream exhibits a carbon intensity score less than 4.0 kg CO₂ eq/kg H₂.

In some embodiments, a method includes receiving, by a pressure swing adsorption (PSA) device, feedstock from a geologic hydrogen source, wherein the feedstock includes geologic hydrogen; producing, by the PSA device, a PSA device effluent and a purge gas stream, the PSA device effluent including hydrogen and helium, and the purge gas stream including unrecovered hydrogen and remaining feedstock components including nitrogen or carbon dioxide or methane; directly or indirectly receiving, by a reactive membrane, the PSA device effluent from the PSA device, wherein the reactive membrane includes a metal selected from the group consisting of palladium, a palladium alloy, platinum, rhodium, ruthenium, copper, nickel, silver, titanium, or another metal; removing, by the reactive membrane, hydrogen from the PSA device effluent; and producing, by the reactive membrane, a first gas stream including predominantly hydrogen and a second gas stream including predominantly helium; wherein the PSA device and the reactive membrane are fluidically coupled to each other.

In some embodiments, the method further includes compressing the feedstock by a compressor fluidically coupled to the geologic hydrogen source. In some embodiments, the PSA includes one or more layers of one or more adsorbent materials from the list of silica gel, alumina, various zeolites, or activated carbons.

In some embodiments, the first gas stream has a hydrogen molar fraction of at least about 90 mol %. In some embodiments, the hydrogen molar fraction is at least about 95 mol %. In some embodiments, the hydrogen molar fraction is at least about 97 mol %. In some embodiments, the hydrogen molar fraction is at least about 99 mol %.

In some embodiments, the second gas stream has a helium molar fraction of at least about 90 mol %. In some embodiments, the helium molar fraction is at least about 95 mol %. In some embodiments, the helium molar fraction is at least about 97 mol %. In some embodiments, the helium molar fraction is at least about 99 mol %.

In some embodiments, the first gas stream has a hydrogen molar fraction of at least about 90 mol %, and wherein the second gas stream has a helium molar fraction of at least about 90 mol %.

In some embodiments, the method further includes powering a power generation plant fluidically coupled to the geologic hydrogen source and to the PSA device with the purge gas stream from the PSA device or the feedstock from the geologic hydrogen source; and providing, by the power generation plant, energy to one or more of a compressor, the PSA device, or the reactive membrane.

In some embodiments, the power generation plant is powered by the feedstock from the geologic hydrogen source only. In some embodiments, the method further includes directing excess power that is not consumed by the compressor, the PSA device, or the reactive membrane to an independent system.

In some embodiments, the reactive membrane is positioned downstream of the PSA device and configured to receive a PSA effluent including hydrogen and helium.

In some embodiments, the reactive membrane is configured to produce (a) a helium-enriched permeate having a helium molar fraction greater than 90 mol %, and (b) a hydrogen-enriched retentate having a hydrogen molar fraction greater than 95 mol %. In some embodiments, the PSA device effluent of the PSA device includes hydrogen and helium.

In some embodiments, the method further includes receiving, by a separation device, the PSA device effluent; producing, by the separation device, a separation device effluent and a purge gas stream, the separation device effluent including hydrogen and helium, wherein the reactive membrane is configured to receive the separation device effluent.

In some embodiments, production of the first gas stream exhibits a carbon intensity score less than 4.0 kg CO₂ eq/kg H₂.

In some embodiments, a system includes purification equipment fluidically coupled to a geologic hydrogen source and configured to receive feedstock from the geologic hydrogen source and produce a hydrogen gas product; and a power generation plant fluidically coupled to the purification equipment and powered by hydrogen from one of the feedstock, the purification equipment, or purge gas from the purification equipment, wherein the power generation plant has a capacity factor greater than 80%; wherein the power generation plant is configured to provide energy to a datacenter providing crypto-mining operations or artificial intelligence training. In some embodiments, the datacenter is islanded. In some embodiments, the datacenter is not connected to the electrical grid.

In some embodiments, the power generation plant has a capacity factor greater than 90%, greater than 95%, greater than 99%, and/or greater than 99.9%. In some embodiments, the system further includes a compressor fluidically coupled to the geologic hydrogen source and to the purification equipment, wherein the compressor is configured to compress the feedstock. In some embodiments, the system further includes a fuel cell configured to consume the hydrogen gas product and to produce an effluent stream included predominantly of helium. In some embodiments, a plant capacity factor is measured over a trailing 12-month period. In some embodiments, the power generation plant operates in an islanded configuration with on-site hydrogen storage sized for at least 12 hours at average load. In some embodiments, the power generation plant includes one or more of: a generator, an engine, a turbine, an oxycombustion power plant, a reciprocating compressor, a fuel cell, or an aero-derivatives gas turbine.

In some embodiments, the purification equipment further includes a reactive membrane, and wherein the hydrogen powering the power generation plant is entirely or partially sourced from the reactive membrane. In some embodiments, the power generation plant is powered by two or more of the feedstock, the purification equipment, or purge gas from the purification equipment. In some embodiments, the hydrogen received by the power generation plant is included predominantly of hydrogen and helium, and wherein the power generation plant includes a fuel cell configured to burn hydrogen and produce an effluent stream included predominantly of helium. In some embodiments, production of the hydrogen gas product by the purification equipment exhibits a carbon intensity score less than 4.0 kg CO₂ eq/kg H₂.

In some embodiments, a method of producing a hydrogen gas product is provided. The method includes processing feedstock using purification equipment fluidically coupled to a geologic hydrogen source; producing, by the purification equipment, the hydrogen gas product; receiving, by a power generation plant fluidically coupled to the purification equipment, hydrogen from one of the feedstock, the purification equipment, or purge gas from the purification equipment, wherein the power generation plant has a capacity factor greater than 80%; and providing, by the power generation plant, energy to a datacenter providing crypto-mining operations or artificial intelligence training. In some embodiments, the datacenter is islanded. In some embodiments, the datacenter is not connected to the electrical grid. In some embodiments, the power generation plant has a capacity factor greater than 90%, greater than 95%, greater than 99%, and greater than 99.9%.

In some embodiments, the method, further includes compressing the feedstock by a compressor fluidically coupled to the geologic hydrogen source and to the purification equipment. In some embodiments, a fuel cell consumes the hydrogen gas product and produces an effluent stream included predominantly of helium. In some embodiments, a plant capacity factor is measured over a trailing 12-month period. In some embodiments, the power generation plant operates in an islanded configuration with on-site hydrogen storage sized for at least 12 hours at average load.

In some embodiments, the power generation plant includes one or more of: a generator, an engine, a turbine, an oxycombustion power plant, a reciprocating compressor, a fuel cell, and an aero-derivatives gas turbine. In some embodiments, the purification equipment further includes a reactive membrane, and wherein the hydrogen powering the power generation plant is entirely or partially sourced from the reactive membrane. In some embodiments, the power generation plant is powered by two or more of the feedstock, the purification equipment, or purge gas from the purification equipment. In some embodiments, the hydrogen received by the power generation plant is included predominantly of hydrogen and helium, and wherein the power generation plant includes a fuel cell configured to burn hydrogen and to produce an effluent stream included predominantly of helium.

In some embodiments, production of the hydrogen gas product by the purification equipment exhibits a carbon intensity score less than 4.0 kg CO₂ eq/kg H₂.

In some embodiments, a system includes a hydrogen source configured to provide a feedstock; first-stage purification equipment fluidically coupled to the hydrogen source and including one or more of: a pressure swing adsorption (PSA) device; a guard bed; a separation membrane; a reactive membrane; or a cryogenic separation device, wherein the first-stage purification equipment is configured to receive the feedstock from the hydrogen source and produce a hydrogen gas product and a carbon dioxide stream, wherein the hydrogen gas product includes hydrogen and helium; an ammonia synthesis loop fluidically coupled to the first-stage purification equipment and configured to receive the hydrogen gas product from the first-stage purification equipment and produce an ammonia product and an effluent; and a urea production unit, wherein the urea production unit is configured to receive an ammonia product from the ammonia synthesis loop and the carbon dioxide stream from the first-stage purification equipment and produce a further ammonia product.

In some embodiments, the hydrogen source is a geologic hydrogen source. In some embodiments, the carbon dioxide stream provided to the urea reactor is derived from PSA purge of the first-stage purification equipment. In some embodiments, the system further includes second-stage purification equipment configured to receive an effluent from the ammonia synthesis loop and produce a helium gas product.

In some embodiments, the system further includes intermediate purification equipment fluidically coupled to the first-stage purification equipment and to the ammonia synthesis loop, wherein the intermediate purification equipment is configured to process the hydrogen gas product from the first-stage purification equipment and to direct an effluent to the ammonia synthesis loop, and wherein the intermediate purification equipment includes one or more of: a PSA device; a guard bed; a separation membrane; a reactive membrane; or a cryogenic separation device.

In some embodiments, the system further includes a compressor fluidically coupled to the hydrogen source and to the first-stage purification equipment, wherein the compressor is configured to compress the feedstock. In some embodiments, purge gas from the first-stage purification equipment is directed to the ammonia synthesis loop. In some embodiments, production of the hydrogen gas product by the first-stage purification equipment exhibits a carbon intensity score less than 4.0 kg CO₂ eq/kg H₂.

In some embodiments, a method of producing a hydrogen gas product is provided. The method includes receiving feedstock from a hydrogen source; processing, by first-stage purification equipment fluidically coupled to the hydrogen source, the feedstock; producing, by the first-stage purification equipment, the hydrogen gas product and a carbon dioxide stream; receiving, by an ammonia synthesis loop fluidically coupled to the first-stage purification equipment, the hydrogen gas product from the first-stage purification equipment; producing, by the ammonia synthesis loop, an ammonia product and an effluent; receiving, by a urea production unit fluidically coupled to the ammonia synthesis loop, the effluent from the ammonia synthesis loop and the carbon dioxide stream from the first-stage purification equipment; and producing, by the urea production unit, a further ammonia product.

In some embodiments, the method further includes compressing the feedstock by a compressor fluidically coupled to the hydrogen source and to the first-stage purification equipment. In some embodiments, the hydrogen source is a geologic hydrogen source. In some embodiments, the carbon dioxide gas stream provided to the urea reactor is derived from PSA purge of the first-stage hydrogen purification equipment. In some embodiments, the method further includes second-stage purification equipment configured to receive an effluent from the ammonia synthesis loop and produce a helium gas product.

In some embodiments, the method further includes processing, by intermediate purification equipment fluidically coupled to the first-stage purification equipment and to the ammonia synthesis loop, the hydrogen gas product from the first-stage purification equipment; and directing, by the intermediate purification equipment, an effluent to the ammonia synthesis loop, wherein the intermediate purification equipment includes one or more of: a PSA device; a guard bed; a separation membrane; a reactive membrane; or a cryogenic separation device.

In some embodiments, the method further includes directing purge gas from the first-stage purification equipment to the ammonia synthesis loop.

In some embodiments, producing the hydrogen gas product by the first-stage purification equipment exhibits a carbon intensity score less than 4.0 kg CO₂ eq/kg H₂.

In some embodiments, a system for producing a hydrogen gas product is provided. The system includes purification equipment fluidically coupled to a geologic hydrogen source, the purification equipment including a guard bed and a pressure swing adsorption (PSA) device, wherein the PSA device is configured to produce the hydrogen gas product, and wherein the purification equipment is not configured to remove helium; wherein the purification equipment is configured to receive feedstock from a geologic hydrogen source and produce the hydrogen gas product, and wherein the hydrogen gas product includes hydrogen and helium; and wherein production of the hydrogen gas product exhibits a carbon intensity score less than 4.0 kg CO₂ eq/kg H₂.

In some embodiments, the PSA device is configured to produce a purge gas including nitrogen, carbon dioxide, methane, and water.

In some embodiments, producing a hydrogen gas product, the system including: receiving, by purification equipment fluidically, feedstock from a geologic hydrogen source, the purification equipment including a guard bed and a pressure swing adsorption (PSA) device, wherein the purification equipment is not configured to remove helium; and producing, by the PSA device, the hydrogen gas product, wherein the hydrogen gas product includes hydrogen and helium; wherein production of the hydrogen gas product exhibits a carbon intensity score less than 4.0 kg CO₂ eq/kg H₂.

In some embodiments, the PSA device is configured to produce a purge gas including nitrogen, carbon dioxide, methane, and water.

In further embodiments, any of the features, functionality and alternatives described in connection with any one or more of FIGS. 1 to 14 may be combined with any of the features, functionality and alternatives described in connection with any other of FIGS. 1 to 14.

The foregoing brief summary is provided merely for purposes of summarizing some example embodiments described herein. Because the above-described embodiments are merely examples, they should not be construed to narrow the scope of this disclosure in any way. It will be appreciated that the scope of the present disclosure encompasses many potential embodiments in addition to those summarized above, some of which will be described in further detail below.

### BRIEF DESCRIPTION OF THE FIGURES

Having described certain example embodiments in general terms above, reference will now be made to the accompanying drawings, which are not necessarily drawn to scale. Some embodiments may include fewer or more components than those shown in the figures.

FIG. 1 is a schematic illustration of an example hydrogen production system including purification equipment that produces a hydrogen gas product having a low carbon intensity score in accordance with some example embodiments described herein.

FIGS. 2, 3, and 3A are schematic flowcharts of exemplary purification equipment of the hydrogen production systems described herein.

FIGS. 4 and 5 are schematic illustrations of example hydrogen production systems that produce a hydrogen gas product having a low carbon intensity score and generate power using feedstock or purge gas in accordance with some example embodiments described herein.

FIGS. 6 and 7 are schematic illustrations of example hydrogen production systems that produce a hydrogen gas product and a helium gas product, each having a low carbon intensity score, in accordance with some example embodiments described herein.

FIGS. 8A-8D and 9 are schematic illustrations of downstream portions of example hydrogen production systems that include an ammonia synthesis loop that produces an ammonia product and second stage purification equipment that produces a helium gas product.

FIG. 10 illustrates a process flow diagram of a baseline case for analysis of an example of a hydrogen production system of the present application.

FIG. 11 is a graph showing production-weighted mean emissions for the baseline case of FIG. 10, including ranges and trends over time.

FIG. 12 is a graph showing year 1 emissions sources by type of the baseline case of FIG. 10.

FIG. 13 is a graph showing sensitivity cases for the baseline case of FIG. 10.

FIG. 14 is a graph showing variation in GHG intensity as a function of hydrogen concentration.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

As used herein, and unless the context dictates otherwise, the following terms have the meanings as specified below.

The term “subsurface hydrogen” refers to any hydrogen in the subsurface, regardless of how the hydrogen came to be in the subsurface.

Hydrogen that is injected through human involvement into the subsurface and retained in the subsurface will be referred to herein as “geologically stored hydrogen.”

In contrast, “geologic hydrogen” refers to any hydrogen in the subsurface that is not geologically stored hydrogen.

The term “natural hydrogen” refers to geologic hydrogen generated in the subsurface without human involvement. Examples of natural hydrogen include hydrogen produced in situ without human prompting by abiotic processes, such as redox reactions (e.g., serpentinization), radiolysis, (geologic) pyrolysis, graphitization, or coalification. Other examples include hydrogen produced in situ without human prompting by biological processes.

In contrast, the term “induced hydrogen” refers to geologic hydrogen created in the subsurface through human involvement, such as hydrogen created by human-prompted injection of a fluid into the subsurface or hydrogen created through other human-prompted alterations to the subsurface.

Geologic hydrogen includes both natural hydrogen and induced hydrogen.

“Enhanced hydrogen generation” refers to the process of creating induced hydrogen through human-prompted techniques primarily intended to promote the in situ formation of hydrogen.

The term “geologic hydrogen derivatives” generally refers to compounds that include hydrogen and are formed from geologic hydrogen, including reactions with fluids, such as air, or other materials introduced from the drilling or sampling process. Example geologic hydrogen derivatives include ammonia, water vapor, water isotopes, and hydrogen cyanide formed from geologic hydrogen.

The term “geologic hydrogen source” generally refers to any system that can produce geologic hydrogen. For example, a wellhead from which geologic hydrogen may be retrieved is a component of a geologic hydrogen source.

The term “hydrogen resource” refers to any system that has the potential to generate hydrogen, has previously generated hydrogen, and/or has the potential to store hydrogen.

The term “geological hydrogen resource” refers to any geological formation that comprises a hydrogen resource.

As used herein, “hydrogen gas product” means a gas delivered from the purification equipment to a product header or discharge port for sale, storage, or downstream use; the product may be a mixture comprising hydrogen (H₂) and helium (He).

Unless otherwise stated, molar fractions are on a dry basis at 0° C. and 1 atm and are measured by gas chromatography.

As used herein, “predominant” means collectively equal to or greater than 90 mol % unless otherwise stated.

As used herein, “majority” means equal to or greater than 50 mol % of the referenced constituent; in some embodiments majority may be equal to or greater than 70 mol %, 80 mol %, or 90 mol %.

The terms “feedstock” or “hydrogen feedstock” generally refers to the gas containing elevated levels of hydrogen received from a geological hydrogen source. In the embodiments described herein, the feedstock of the geological hydrogen source includes hydrogen and helium.

### Overview

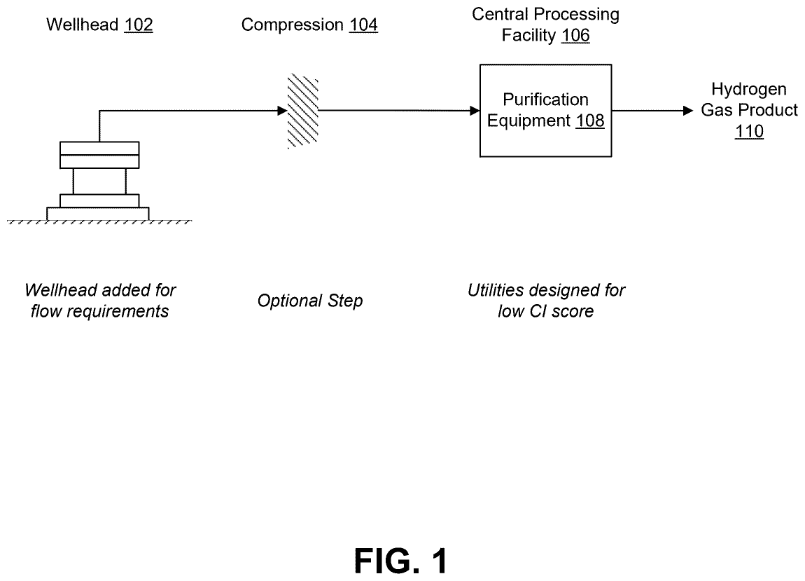

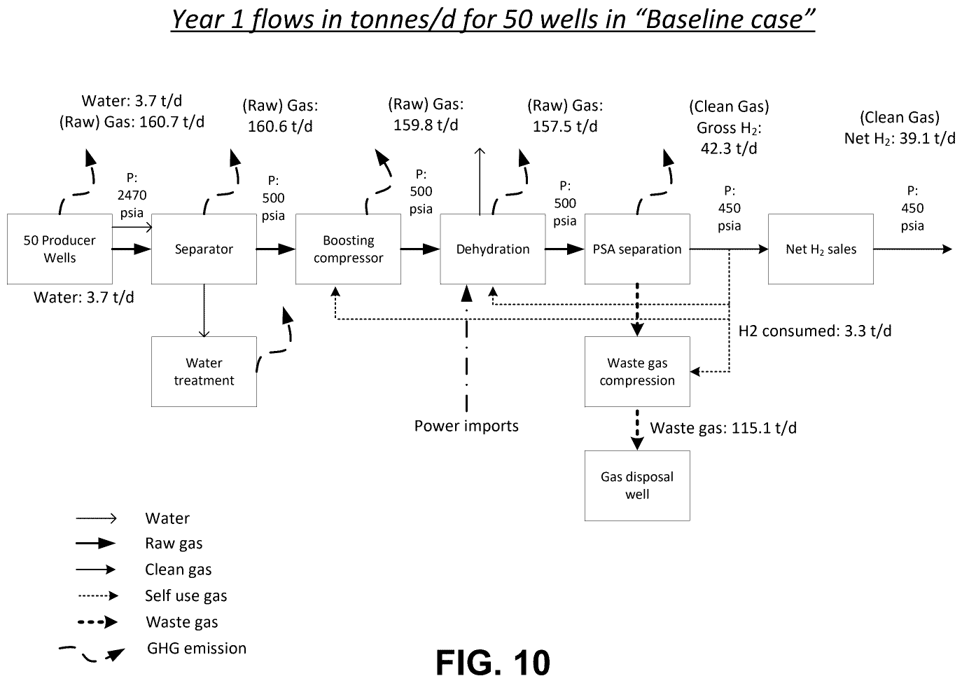

FIGS. 1-9 illustrate exemplary flow diagrams of hydrogen production systems that produce a hydrogen gas product having a low CI score, and FIGS. 10-13 provide flowcharts and data related to a modeled hydrogen production system in accordance with the embodiments described herein. The CI scores referenced herein are provided in kg CO₂ equivalent greenhouse gases per kg H₂ produced (kg CO₂ eq/kg H₂), and the calculation thereof is described in detail with respect to the modeled hydrogen production system described below. The CI score of the hydrogen gas product is influenced by the gas composition of the feedstock from the geologic hydrogen source as well as the well depth, productivity, and other parameters. The CI score is also minimized when the hydrogen production system powers and heats the purification equipment and other components with low-carbon energy sources, such as self-produced hydrogen.

In at least one embodiment, the hydrogen gas product is a mixed stream in which hydrogen and helium are the predominant constituents. For example, the hydrogen gas product includes a combined hydrogen and helium molar fraction of equal to or greater than 98 mol %, optionally 99 mol %, and the nitrogen molar fraction is equal to or greater than 2 mol % (measured on a dry basis at standard temperature and pressure).

In certain embodiments, the hydrogen gas product comprises hydrogen and helium in the same stream with the helium molar fraction equal to or greater than 0.1 mol %, 1.0 mol %, or 2.0 mol %.

In some embodiments, the purification equipment does not include a component configured to remove helium. More specifically, in some embodiments, the purification equipment is not configured to remove helium. In one embodiment, the purification equipment includes a PSA device and does not include a component configured to remove helium downstream of the PSA device. In other embodiments, the purification equipment includes a PSA device and does not include a component configured to remove helium upstream of the PSA device. In a still further embodiment, the purification equipment includes a PSA device and does not include a component configured to remove helium at a point between the PSA device and a hydrogen gas product outlet.

In certain embodiments, a controller adjusts the purge rate, equalization timing, or step durations to maintain a target helium content in the hydrogen gas product of, for example, 0.1-5 mol %, while meeting hydrogen recovery targets.

Illustrative operating ranges include: a feed pressure of 300-1,500 psig, preferably 500-1,000 psig; a feed temperature of 0-60° C.; a PSA cycle time of 30-300 s; 4-12 guard beds with pressure equalization; a purge fraction of 1-10%; and hydrogen recovery of 60-92%.

Examples of guard-bed media include ZnO, Cu/Zn, 13X, 5A, and activated carbon; examples of PSA adsorbents include 5A, 13X, and carbon molecular sieve (CMS). Helium tends to track with hydrogen through PSA due to weak adsorption and low polarizability.

### Carbon Intensity (CI) Framework

Carbon intensity (CI) is expressed as kg CO₂ eq/kg H₂ at the plant gate, or point of production, and is calculated with system boundaries covering Scope 1 and Scope 2 carbon emissions, referring to direct and indirect greenhouse gas emissions caused by the hydrogen production system as categorized by the U.S. EPA. Electricity emission factors are obtained from a disclosed, dated source; gas compression energy is modeled on a per-unit-operation basis using engineering estimates or metered values when available.

Unless noted, co-produced helium is treated using system expansion or is assigned no credit; sensitivity cases may allocate credit to evaluate robustness. Upstream methane leakage may occur.

Representative CI targets include less than 4.0 kg CO₂ eq/kg H₂ (baseline), less than 3.0 kg CO₂ eq/kg H₂ (preferred), less than 1.5 kg CO₂ eq/kg H₂ (preferred) and less than 0.45 kg CO₂ eq/kg H₂ (exemplary, e.g., with low-carbon electricity and heat integration).

In the present application, the production of the hydrogen gas product provided by the hydrogen production systems and methods described herein exhibits a carbon intensity score less than 3.0 kg CO₂ eq/kg H₂. In other embodiments, the carbon intensity score is less than 1.5 kg CO₂ eq/kg H₂, preferably less than 0.45 kg CO₂ eq/kg H₂, and more preferably less than 0.37 kg CO₂ eq/kg H₂. The low CI score is achieved by using a starting material having a minimum hydrogen molar fraction of at least 50 mol %, the ordering of purification equipment to minimize power consumption and efficiently improve purity, and the use of a power generation plant that is powered by hydrogen or purge gas from purification equipment and provides power to equipment within the hydrogen production system.

### Geologic Hydrogen Source

In the embodiment illustrated in FIG. 1, feedstock is captured at a geologic hydrogen source, such as a wellhead 102 that captures subsurface gas from a wellbore at least partially traversing a rock formation. The wellbore provides a pathway for the recovery of fluids or feedstock therefrom. Generally, rock deposits yield abiotic hydrogen through the reaction of water with the rock deposits to mineralize oxygen and release hydrogen, such as in the serpentinization reaction or radiolysis. In some example embodiments, a two-step reaction is utilized that first generates hydrogen through the injection of a water-based stimulant into the wellbore, and then mineralizes oxygen into the rock formation while liberating hydrogen. Example embodiments can achieve hydrogen recovery by identifying rock formations having suitable characteristics, subsurface depths that optimize the preferred chemical reactions of fluids with rock, and the sequencing and nature of the recovery.

In some embodiments, the wellhead 102 collects feedstock from at least about 300 feet below ground level. In other embodiments, the wellhead collects feedstock from at least about 1,000 feet below ground level. In still further embodiments, the wellhead collects feedstock from between about 2,000 and about 3,000 feet below ground level, or between about 3,000 and about 4,000 feet below ground level, between about 4,000 and about 5,000 feet below ground level, between about 5,000 and about 6,000 feet below ground level, between about 6,000 and about 12,000 feet below ground level, or from at least about 20,000 feet below ground level.

In still further embodiments, the wellhead 102 demonstrates a productivity of at least 1.33 billion standard cubic feet per well. In other embodiments, the well demonstrates a productivity greater than 250 tonnes of hydrogen per year. In still further embodiments, the well demonstrates a productivity greater than 500 tonnes of hydrogen per year, 1,000 tonnes of hydrogen per year, 2,000 tonnes of hydrogen per year, 3,000 tonnes of hydrogen per year, 4,000 tonnes of hydrogen per year, 5,000 tonnes of hydrogen per year, 6,000 tonnes of hydrogen per year, 7,000 tonnes of hydrogen per year, 8,000 tonnes of hydrogen per year, 9,000 tonnes of hydrogen per year, or 10,000 tonnes of hydrogen per year.

In some embodiments, the feedstock includes hydrogen, helium, and nitrogen. A hydrogen molar fraction of the feedstock may be at least about 50 mol %. In other embodiments, the hydrogen molar fraction may be at least about 60 mol %, at least about 70 mol %, at least about 80 mol %, or at least about 85 mol %. In some embodiments, a helium molar fraction of the feedstock may be greater than about 0.1 mol %, about 0.5 mol %, about 1 mol %, about 1.5 mol %, about 2 mol %, about 2.5 mol %, about 3 mol %, or about 5 mol %. A nitrogen molar fraction of less than about 20 mol %, about 15 mol %, about 12 mol %, about 10 mol %, about 8 mol %, about 5 mol %, or about 3 mol %.

The feedstock may also have a methane molar fraction of less than about 1.5 mol % or less than about 1 mol %. The feedstock may have a methane concentration of less than about 10,000 ppm, or preferably less than 1,000 ppm. The feedstock may also have less than about 10,000 ppm hydrocarbons, less than about 1,000 ppm hydrocarbons, or a hydrocarbon molar fraction of up to about 1 mol %.

In some embodiments, the geologic hydrogen source includes one wellhead 102, while in other embodiments, the geologic hydrogen source includes a plurality of wellheads 102. Still further, in certain embodiments, the geologic hydrogen source may be free from hydrogen that is produced using electrolysis, steam methane reformation, methane pyrolysis, or gasification. In other embodiments, the hydrogen production system may receive hydrogen feedstock from other sources in addition to the feedstock from a geologic hydrogen source.

As shown in FIGS. 1 and 4-9, the geologic hydrogen source may be fluidically coupled to a compressor 104, and the feedstock may initially be directed to a compressor 104 to increase the pressure of the feedstock prior to treatment. As used herein, elements described as being “fluidically coupled” are coupled together with an appropriate line or other means to permit the passage of gas therebetween. In the case of multiple wellheads, each wellhead 102 may be fluidically coupled to a respective compressor 104, such that the individual feedstock streams from each wellhead 102 may be directed to a respective compressor 104 before being combined into a single feedstock. In other embodiments, multiple wellheads 102 may be fluidically coupled to one or more compressors 104, such that multiple feedstock streams from multiple wellheads 102 are directed to an arrangement of one or more compressors 104 prior to treatment by a central processing facility 106 that includes purification equipment 108. The one or more compressors 104 may in turn be fluidically coupled to the central processing facility 106 (e.g., to purification equipment 108). Accordingly, the geologic hydrogen source may be fluidically coupled to the central processing facility 106, either indirectly via one or more compressor 104, or directly in embodiments where no compressor 104 is required.

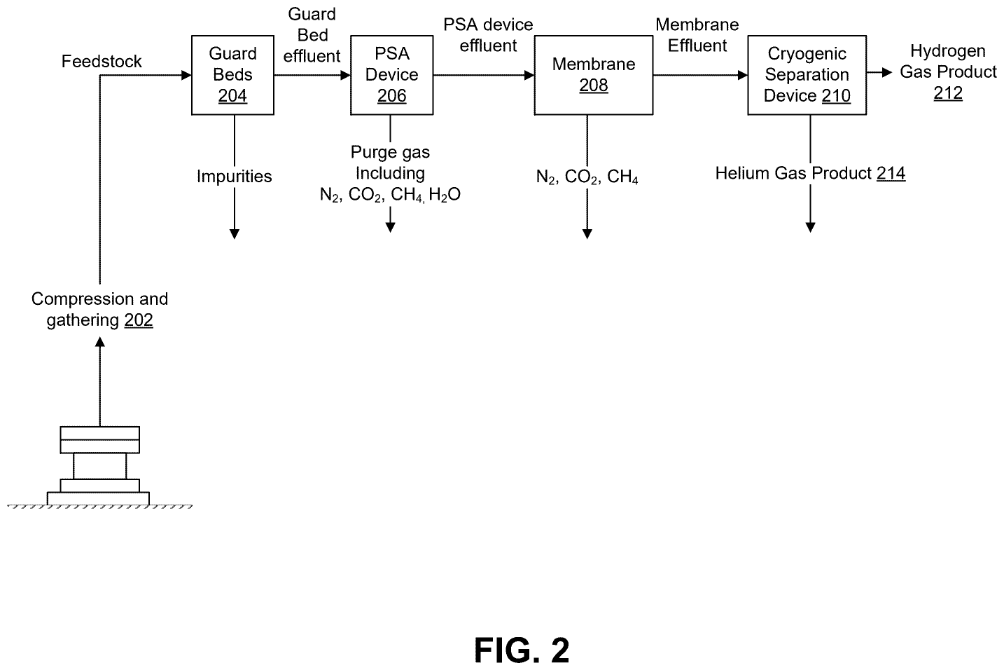

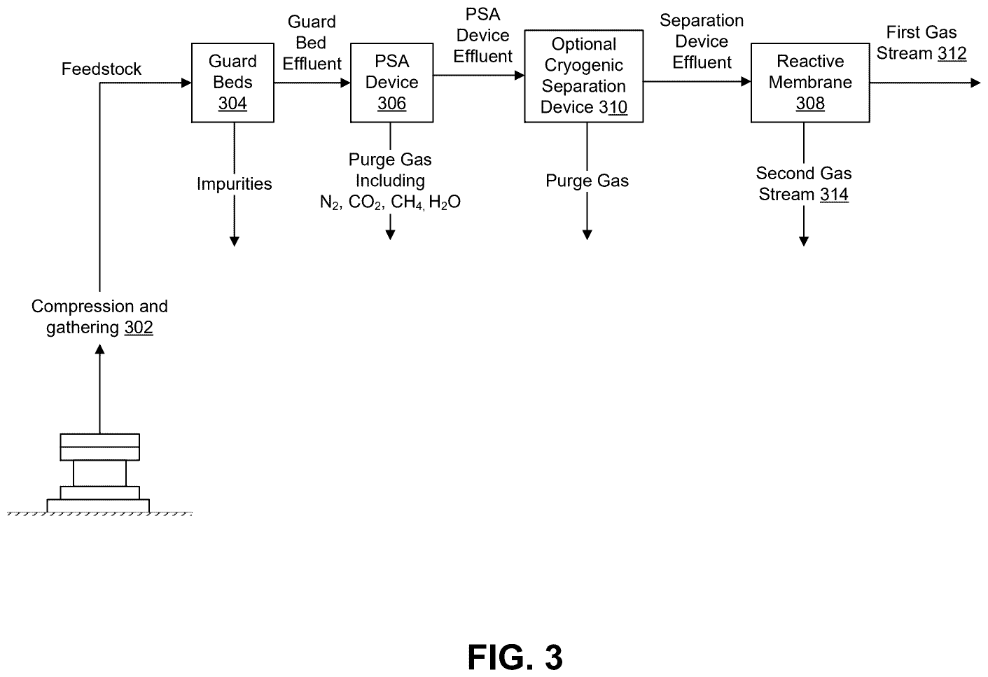

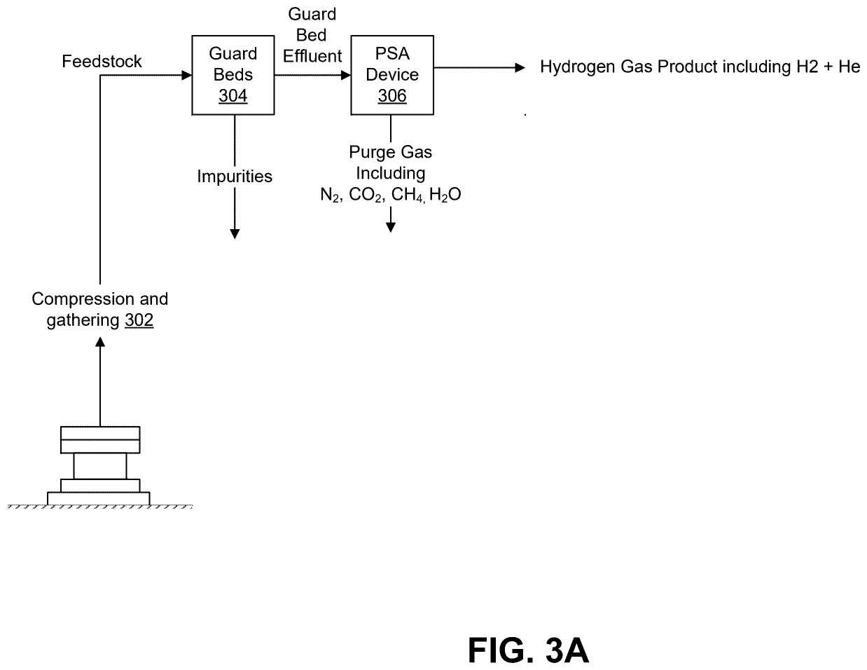

Purification Equipment The purification equipment 108 of the central processing facility 106 separates specific gas components of the feedstock, purifies the hydrogen to produce a hydrogen gas product 110 (e.g., sale gas), and/or purifies helium or other noble gases to produce a noble gas product. The purification equipment 108 includes components (which, in various embodiments, may be fluidically coupled to each other) that remove impurities, hydrocarbons, nitrogen, and noble gases. FIGS. 2 and 3 illustrate example purification flow diagrams, although the ordering of the purification equipment may be modified or altered.

As shown in FIGS. 2-3, the feedstock may be received by the central processing facility 106 following compression and gathering 202, 302 (e.g., as described previously), For example, the purification equipment 108 may include one or more guard beds or reactor beds 204, 304 to remove impurities such as sulfur, particulates, metals, or liquid components. Example guard beds 204, 304 include an activated carbon guard bed, an adsorbent guard bed, a copper oxide guard bed, or a zinc oxide guard bed, although other suitable guard beds may be used depending on the facility needs and particular feedstock composition. The purification equipment 108 may include any number of guard beds 204, 304 needed as required by the productivity of the geologic hydrogen source. Suitable alternatives for a guard bed include, but are not limited to, a drier bed or a knock-out vessel.

The purification equipment 108 may also include a pressure swing adsorption (PSA) device 206, 306 that removes gases such as nitrogen, carbon dioxide, and methane from the feedstock. These components are typically captured in a purge gas, resulting in the feedstock from the geologic hydrogen source, now the effluent from the PSA device 206, 306, including primarily hydrogen and helium. In some embodiments, the feedstock or PSA device effluent has a hydrogen concentration of at least 70 mol %. As described in greater detail below, the purge gas may be reused within the hydrogen production system.

One or more separation membranes 208 may also be included in the purification equipment 108 to remove additional nitrogen, carbon dioxide, and methane. Example separation membranes 208 include spiral wound membranes, a hollow fiber membrane, or a filtration membrane. In some embodiments, the purification equipment 108 may also include a reactive membrane 308. Said separation membranes or reactive membranes may remove an additional 50% of the nitrogen, carbon dioxide, and methane, or an additional 70% of the nitrogen, carbon dioxide, and methane, or an additional 90% of the nitrogen, carbon dioxide, and methane, or an additional 95% of the nitrogen, carbon dioxide, and methane.

Cryogenic separation devices 210 may also be included in the purification equipment 108 to separate hydrogen, helium, and other gases, resulting in high purity gas streams. Example cryogenic separation devices 210 include distillation systems, heat exchangers, separator vessels, valves, expanders, cryogenic distillation columns, a series of cryogenic distillation columns, or the like.

FIGS. 2 and 3 illustrate example purification processes that increase the purity of hydrogen and helium within the feedstock and then produce both a hydrogen gas product 212 and a helium gas product 214. The hydrogen production systems described herein include equipment to capture and produce a helium gas product 214 as the feedstock from the geologic hydrogen source includes a significant and valuable helium component. In the first embodiment illustrated in FIG. 2, the final phase of the purification process is a cryogenic separation device 210, such as a cryogenic distillation column, that provides a hydrogen gas product 212 and a helium gas product 214. In the second embodiment illustrated in FIG. 3, the final phase of the purification process is a reactive membrane 308 that produces a first gas stream 312 comprised predominantly of hydrogen and a second gas stream 314 comprising helium.

In the embodiment of FIG. 2, the feedstock is first directed to one or more guard beds 204 that remove feedstock impurities such as water, carbon dioxide, hydrocarbons, sulfur, particulates, metals, and/or liquid components from the feedstock. The guard bed effluent is then directed to a PSA device 206, which removes components such as water, nitrogen, carbon dioxide, and methane from the guard bed effluent in a purge gas stream. The PSA device may include, for example, layers of adsorbent materials optimally selected to remove said components. Typical adsorbent materials include silica gel, alumina, various zeolites, and activated carbons; a PSA device may include one or more layers of one or more of these adsorbent materials. The PSA device effluent comprises the majority of hydrogen and helium, and the PSA device purge gas stream may comprise a minority of unrecovered hydrogen and helium.

Next, the PSA device effluent is directed to a membrane 208, which could be a separation membrane and/or a reactive membrane. The membrane 208 also removes nitrogen, carbon dioxide, and methane from the feedstock. Then the membrane effluent is directed to a cryogenic separation device 210, such as cryogenic distillation columns. The cryogenic separation device 210 removes helium from the feedstock, producing the hydrogen gas product 212 and the helium gas product 214.

The purification process of FIG. 3 differs from FIG. 2 in that PSA device effluent from the PSA device 306 is directed to a reactive membrane 308 that produces a first gas stream 312 comprised predominantly of hydrogen and a second gas stream 314 comprised predominantly of helium. The process of FIG. 3 may also optionally include a cryogenic separation device 310 upstream of the reactive membrane 308 for separating out gases other than hydrogen and helium. Example cryogenic separation devices 310 include distillation systems, heat exchangers, separator vessels, valves, expanders, cryogenic distillation columns, a series of cryogenic distillation columns, or the like.

The reactive membrane 308 include a membrane within a reactor that in situ separates the hydrogen from the gas stream. The reactive membrane 308 may include, for example, a metallic membrane comprising palladium, a palladium alloy, platinum, rhodium, ruthenium, copper, nickel, silver, titanium, combinations thereof, or another metal. The reactive membrane may also comprise a ceramic material such as alumina, silica, or zirconia.

For example, the reactive membrane may include one or more metallic or ceramic support layers with a hydrogen permeable thin film membrane covered by a protective layer. The thin film may include palladium, a palladium alloy, platinum, or another suitable material that interacts selectively with hydrogen. The protective layer may also include palladium, a palladium alloy, platinum, rhodium, ruthenium, copper, nickel, silver, titanium, combinations thereof, or another metal.

In some examples, the first gas stream 312 has a hydrogen molar fraction of at least about 90 mol %, 95 mol %, 97 mol %, or 99 mol %. The second gas stream 314 may have a helium molar fraction of at least about 90 mol %, 95 mol %, 97 mol %, or 99 mol %.

In some embodiments, reactive membranes provide an improved performance of removing gases other than hydrogen over separation membranes. The performance of the reactive membrane may depend in part on the components upstream thereof. For example, processing the feedstock through a PSA device 306 prior to treatment in the reactive membrane 308 removes certain gases such as nitrogen, carbon dioxide, and methane such that the reactive membrane 308 can more efficiently remove hydrogen from the gas stream. Further, processing the feedstock from the PSA device 306 through a cryogenic separation device 310 prior to treatment in the reactive membrane 308 to remove gases other than hydrogen and helium may also improve performance of the reactive membrane.

In some embodiments, a size selective membrane may selectively remove helium as well as hydrogen from the rest of the components of an incoming gas stream. In some embodiments said size selective membrane can be used in conjunction with a PSA, being positioned on the effluent gas stream comprising hydrogen and helium, coming out of a PSA to further increase the concentration of said hydrogen and helium.

FIG. 3A is a schematic diagram of an example system configured to produce a hydrogen gas product from a geologic hydrogen source. The system includes a guard bed and a pressure swing adsorption (PSA) device coupled in series. In some embodiments, the hydrogen gas system includes a plurality of guard beds 304 and/or plurality of PSA devices 306. A geologic feedstock comprising hydrogen, helium, nitrogen, carbon dioxide, methane, and other inert gases is provided to the guard bed 304 for contaminant removal and then to the PSA device 306. The PSA device 306 produces (i) a hydrogen gas product that comprises hydrogen and helium in the same gas stream and (ii) a purge stream that includes unrecovered hydrogen and non-hydrogen components such as nitrogen, carbon dioxide, and methane. In some embodiments, the purge gas may include unrecovered helium.

### Power Generation

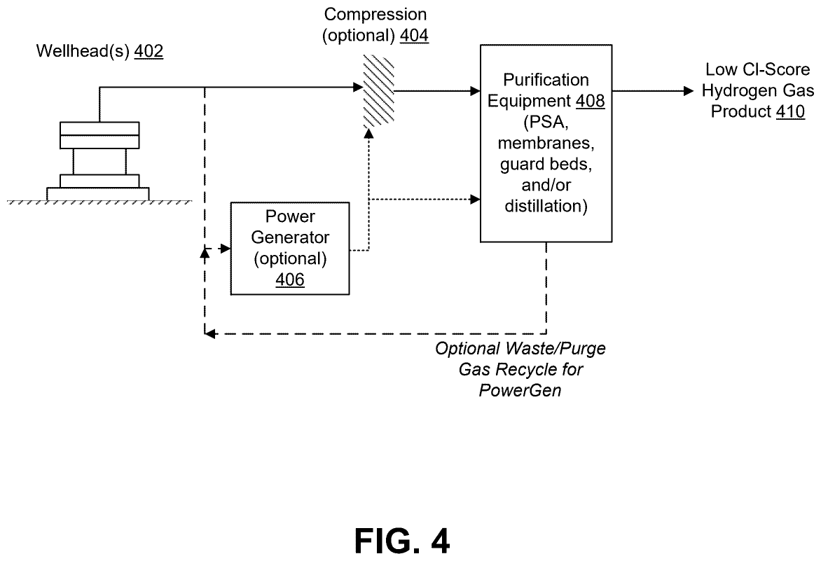

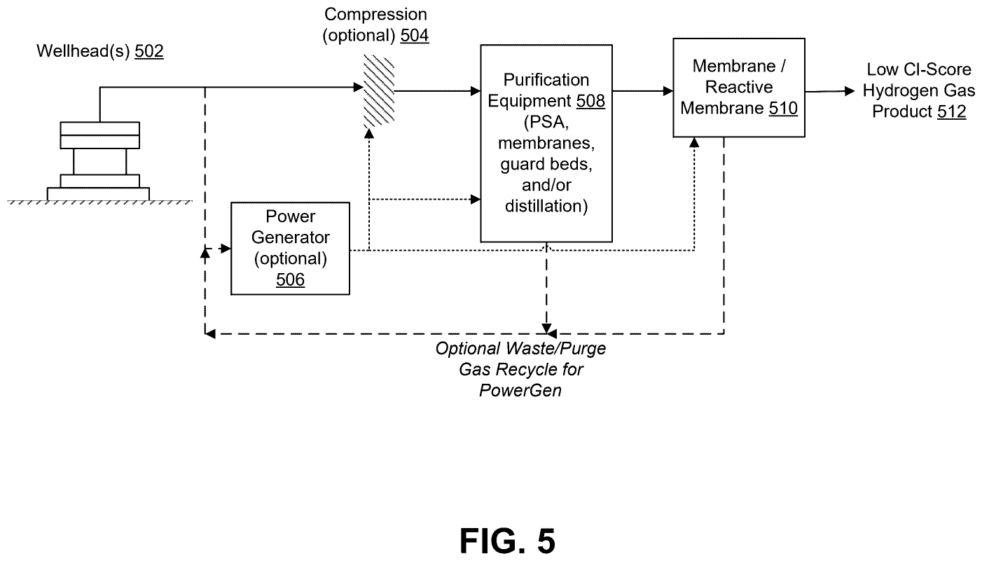

In further embodiments such as the embodiments illustrated in FIGS. 4 and 5, the hydrogen production system also includes a power generation plant 406, 506 that is powered by hydrogen from either of the feedstock from the geologic hydrogen source 402, 502 or the purification equipment 408. In some embodiments, the power generation plant 406, 506 is powered by the feedstock from the geologic hydrogen source 402, 502 only. In such embodiments, operating the power generation plant 406, 506 in this fashion avoids the production of greenhouse gases otherwise required to fuel the power generation plant 406, 506, and thereby further enhances the ability of the hydrogen production system to produce a low-CI score hydrogen gas product 410, 512.

Additionally or alternatively, the power generation plant 406, 506 may be partially or entirely powered by waste or purge gas from one or more of the purification equipment 408, including a reactive membrane 510 (FIG. 5). For example, the reactive membrane is configured to produce (a) a helium-enriched permeate having a helium molar fraction greater than 90 mol % and (b) a hydrogen-enriched retentate having a hydrogen molar fraction greater than 95 mol %. The purge gas may include various components such as unrecovered hydrogen, nitrogen, carbon dioxide, and methane, and can be used to generate power using a hydrogen gas turbine, a hydrogen engine, a hydrogen reciprocating generator, or other suitable power generator. In some embodiments, a PSA and/or a cryogenic device is provided upstream of the reactive membrane 510.

The power generation plant 406, 506 may include a generator, an engine, a turbine, an oxycombustion power plant, a reciprocating generator, a linear generator, a reciprocating compressor, a fuel cell, an aero-derivatives gas turbine, or another suitable power generator. The power generation plant 406, 506 in the hydrogen production system may be powered by hydrogen, purge/waste gas from the purification equipment 408, 508, or feedstock from the geologic hydrogen source 402, 502, in contrast to conventional power generators that are powered by fossil fuels.

Further, the power generation plant 406, 506 may be configured to provide energy to the compressor 404, 504 or components of the purification equipment 408, 508, including a reactive membrane 510 (FIG. 5). In some embodiments, the power generation plant 406, 506 also produces excess or additional energy that is provided to an independent system (not shown). Independent systems may include a power grid, compressors, datacenters, crypto mining operations, artificial intelligence (AI) processors, or other facilities. For example, crypto and AI datacenters that are optionally islanded may be powered by the power generation plant 406, or 506, resulting in a capacity factor of greater than 80%, greater than 90%, greater than 95%, greater than 99%, or greater than 99.9% without utilizing a grid connection. Said islanded facilities may be optionally connected to the electric grid for use as back-up power. In some embodiments, a plant capacity factor is measured over a trailing 12-month period.

Use of the hydrogen production systems described herein providing low CI-score hydrogen as a source for power generation enables the crypto and AI datacenters to operate at with a significant power consumption with minimal impact on the local power grid. Use of the hydrogen production systems described herein providing low CI-score hydrogen as a source for power generation enables the crypto and AI datacenters to operate at with a significant power consumption with minimal impact on carbon emissions.

In another embodiment, the hydrogen received by the power generation plant 406, 506 is comprised predominantly of hydrogen and helium. When the power generation plant 406, 506 is powered by feedstock from the geologic hydrogen source 402, 502, the feedstock includes noble gases that can be separated and captured. For example, the power generation plant 406, 506 may comprise a fuel cell configured to burn hydrogen and produce an effluent stream comprised predominantly of helium as well as generate power. In contrast, power generation plants configured to be powered by hydrogen from other sources such as electrolysis cannot produce an effluent stream comprising a noble gas.

In still further embodiments, the power generation plant may operate in an islanded configuration with on-site hydrogen storage. For example, the storage may be sized for at least 12 hours at average load, while in other embodiments, the storage is sized for a smaller load or a larger load, as desired.

### Noble Gas Production

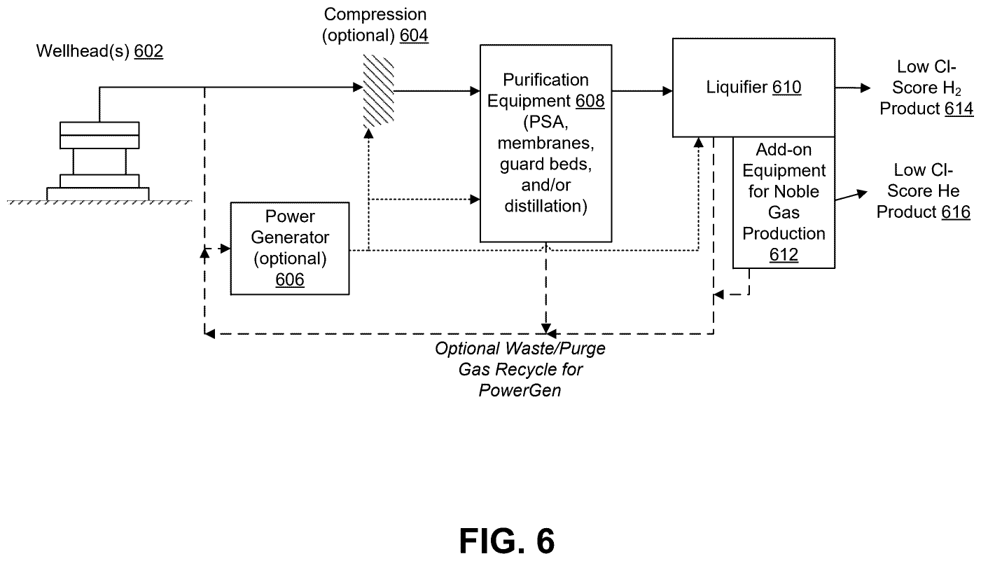

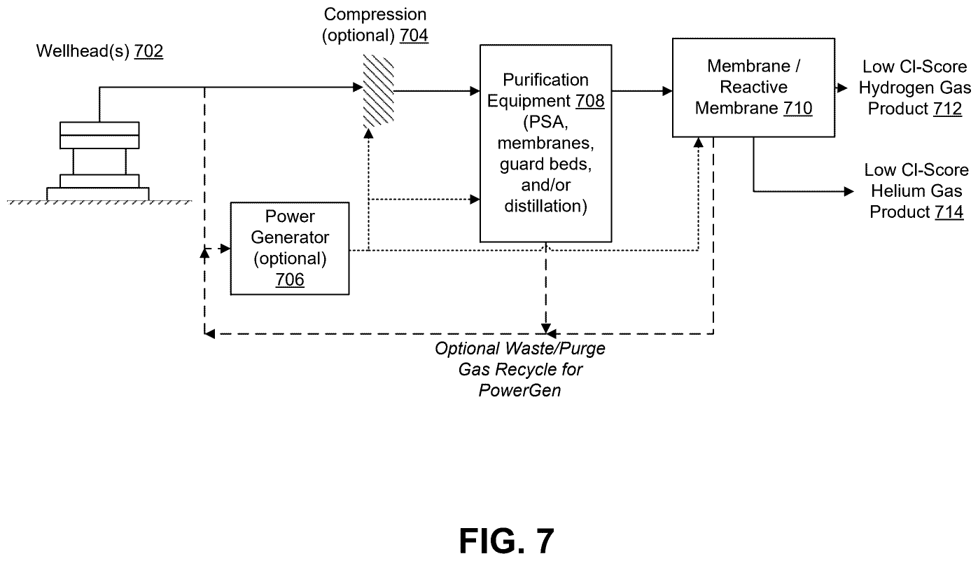

Referring to FIGS. 6 and 7, further embodiments of the hydrogen production system include a purification process for producing a noble gas product 616, 714. In such embodiments, the hydrogen production system includes first-stage purification equipment 608/610, 708 configured to receive feedstock from the geologic hydrogen source 602, 702 (optionally, following compression 604, 704) and produce an effluent, and second-stage purification equipment 612, 710 configured to receive the effluent from the first-stage purification equipment 608, 708 and produce a hydrogen gas product 614, 712 and a noble gas product 616, 714. The hydrogen production system shown in FIGS. 6 and 7 may, optionally, also be powered by a power generator 606, 706, in the manner described in connection with FIG. 5.

In the illustrated embodiments, the noble gas product 616, 714 comprises at least one of helium, neon, argon, xenon, krypton, or radon. In some embodiments, the noble gas product 616, 714 includes helium having a molar fraction of at least about 90 mol %, 95 mol %, 97 mol %, 99 mol %, or 99.5 mol %. In some embodiments, the second-stage purification equipment 612, 710 produces multiple noble gas products 616, 714. For example, the second-stage purification equipment 612, 710 may include a cryogenic separation device that includes a plurality of columns, which would allow for different gases to be captured.

The second-stage purification equipment may include a liquefier (e.g., liquefier 610), a guard bed, a further pressure swing adsorption (PSA) device, a separation membrane, and/or a reactive membrane. In some embodiments, a liquefier separates the noble gas(es) from the hydrogen. In still further embodiments, the liquefier produces the hydrogen gas product 614, 712 and the additional gases separated therefrom are directed to second-stage purification equipment 612, 710 that purifies and produces the noble gas product 616, 714. In still further embodiments, a separation or reactive membrane 208, 308 may be used to separate the noble gas from the hydrogen. In some embodiments additional cryogenic distillation columns reside in the same cold box as the liquefier and further separate the noble gases from each other.

### Downstream Integrations

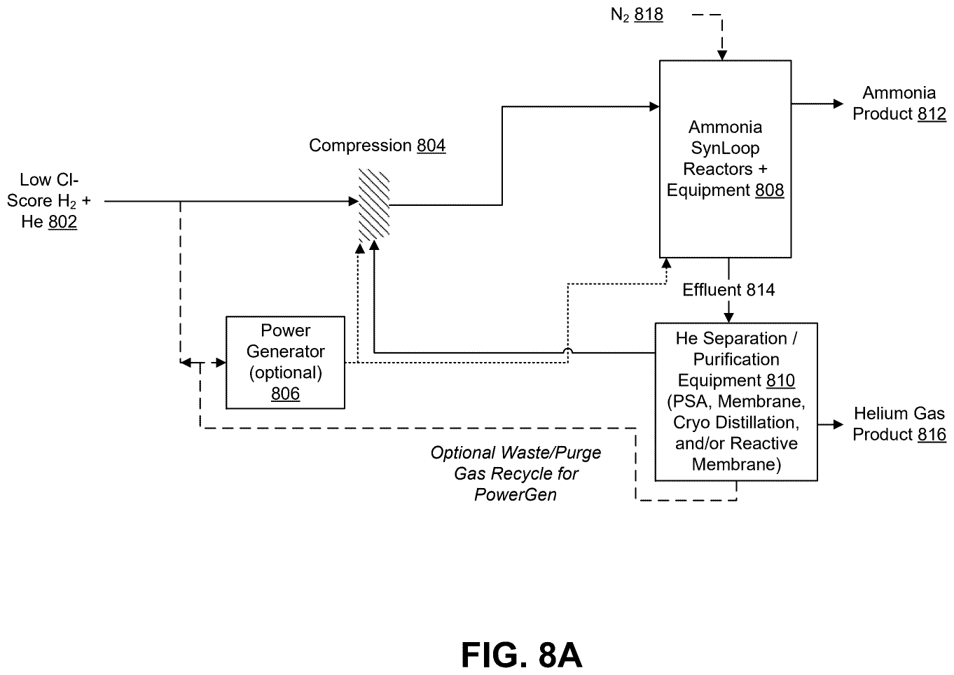

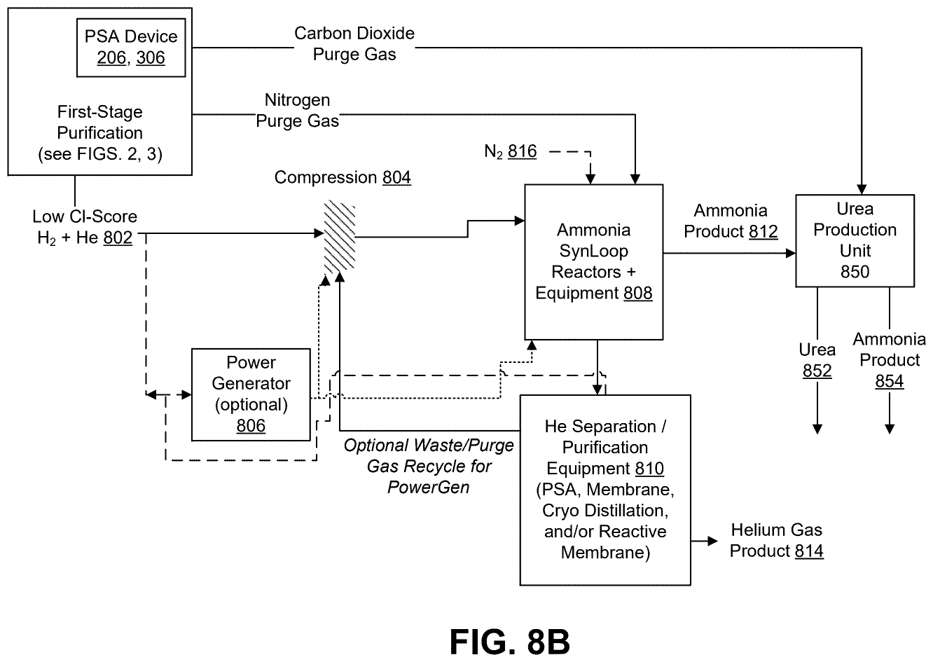

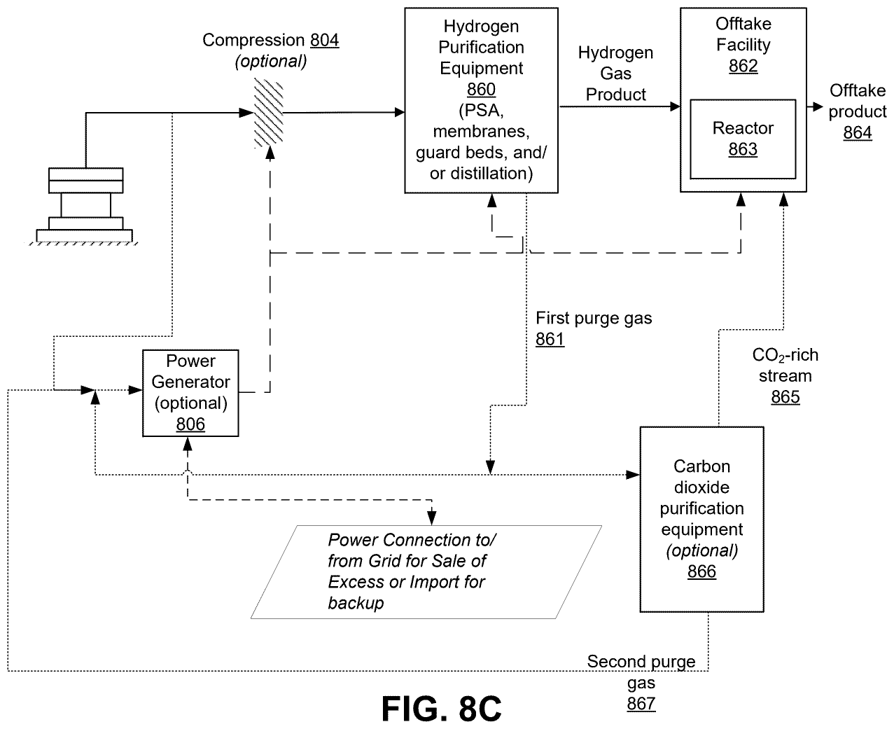

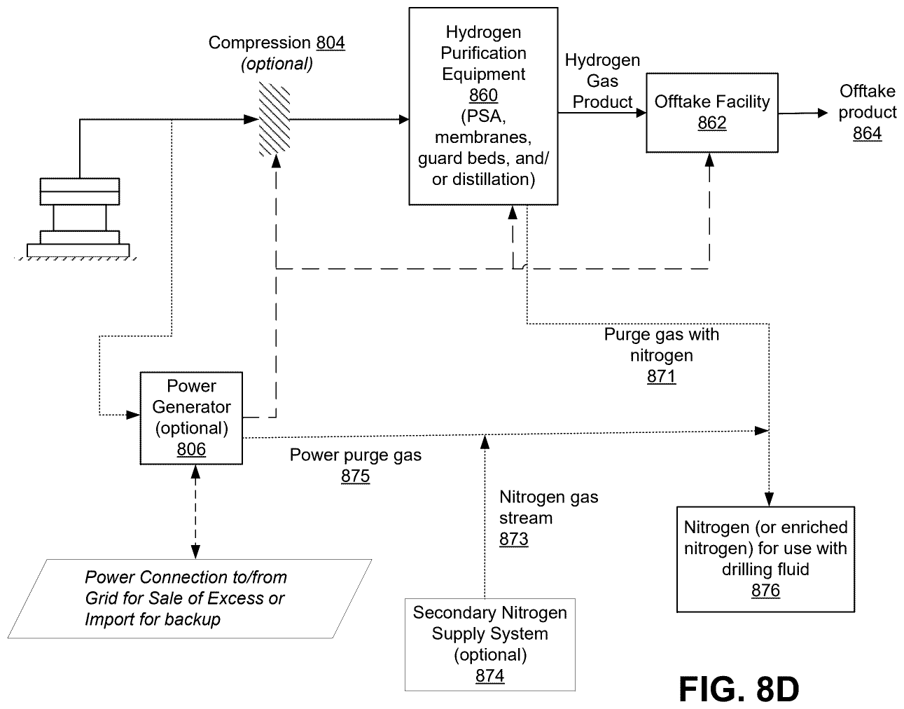

Referring to FIGS. 8A and 8B, the hydrogen production system may also include an ammonia synthesis loop 808 that utilizes the hydrogen gas product 802 from the first-stage purification equipment 108, 408, 508, 608/610, 710 (see FIG. 8B) to produce an ammonia product 812. The hydrogen gas product 802 is first pressurized in a compressor 804 before entering the ammonia synthesis loop 808, which also receives nitrogen from a separate source 818. In other embodiments, the ammonia synthesis loop 808 described herein may be provided separately from the purification equipment 108, 408, 508, 608/610, 710. For example, the hydrogen gas product 802 may be delivered from the purification equipment to the ammonia synthesis loop 808 through a distribution system or other transport (e.g., the purification equipment may be fluidically coupled to the ammonia synthesis loop 808). As shown in FIGS. 8A and 8B, the hydrogen production system may optionally also be powered by a power generator 806 in the manner described in connection with FIG. 5.

The ammonia synthesis loop 808 produces an effluent 814 in addition to the ammonia product 812. The effluent 814 is directed to second-stage purification equipment 810 that produces a noble gas product such as a helium gas product 816. The second-stage purification equipment 810 may include a liquefier, a guard bed, a further pressure swing adsorption (PSA) device, a separation membrane, and/or a reactive membrane.

As shown in FIG. 8B, the ammonia product 812 from the ammonia synthesis loop 808 may be directed to additional components, such as a urea production unit 850. The urea production unit 850 may produce urea or a further ammonia product 852 as well as an ammonia product stream 854 that was not used to generate urea. Other components that utilize ammonia may be included downstream of the ammonia synthesis loop 808 as well. One embodiment of a urea production unit is a reactor wherein the Bosch-Meiser process takes place, ammonia and carbon dioxide react to form ammonia carbamate, which subsequently decomposes to urea and water.

In some embodiments, gases such as carbon dioxide and/or nitrogen removed from the feedstock in the first-stage purification equipment 108, 408, 508, 608/610, 710 may be used in the ammonia synthesis loop 808 or downstream components such as the urea production unit 850. For example, as shown in FIG. 8B, the urea production unit 850 may receive carbon dioxide purge gas from a PSA device 206, 306 of the first-stage purification equipment. Alternatively, or additionally, nitrogen purge gas from the first-stage purification equipment 108, 408, 508, 608/610, 710 may be directed to the ammonia synthesis loop 808 in addition or as an alternative to the nitrogen from a separate source 818.

Carbon dioxide and/or nitrogen removed from the feedstock in the first-stage purification equipment 108, 408, 508, 608/610, 710 may also be used in other downstream components. In the embodiment illustrated in FIG. 8C, the hydrogen production system may also include an offtake facility 862 that utilizes both the hydrogen gas product as well as carbon dioxide sourced from the purge gas 861 of the first-stage or hydrogen purification equipment 860.

The hydrogen purification equipment 860 may include first-stage purification equipment 108, 408, 508, 608/610, 710. In one embodiment, the hydrogen purification equipment 860 includes a PSA device that provides an effluent containing hydrogen as well as a first purge gas 861. In other embodiments, the first purge gas 861 is provided by other purification equipment. The hydrogen purification equipment 860 is configured to provide configured to receive the feedstock from the geologic hydrogen source and produce a hydrogen gas product as described with respect to the embodiments of FIGS. 1-8.

The first purge gas 861 from the hydrogen purification equipment 862 may be directed to optional carbon dioxide or second-stage purification equipment 866, which purifies the first purge gas to provide a carbon dioxide-rich gas stream 865 and a second purge gas 867. In some embodiments, the carbon dioxide-rich gas stream 865 has a carbon dioxide molar fraction of at least 50 mol %. The carbon dioxide purification equipment 866 may be second PSA device, a temperature-swing adsorption, a separation membrane, a chemical-solvent absorption system, or a cryogenic distillation system. Where product quality specifications require a carbon dioxide molar fraction of at least 99 mol %, the carbon dioxide purification equipment 866 may be an amine contactor or caustic wash column. The second purge gas 867 from the carbon dioxide purification equipment 866, which may include unrecovered hydrogen, nitrogen, carbon dioxide, methane, and/or other gases, may be utilized by a power generation plant to generate power for the purification compressors, the hydrogen purification equipment 860, or external third parties such as the grid.

Further, the offtake facility 862 receives the hydrogen gas product from the hydrogen purification equipment 860 as well as the carbon dioxide-rich gas stream 865 from the carbon dioxide purification equipment 866 and produces a synthesis product 864. In some embodiments, the offtake facility 862 includes a reactor(s) 863 that utilize(s) hydrogen and carbon dioxide from the respective streams to generate the synthesis product 864, such as synthetic natural gas, methanol, ethanol, Fischer-Tropsch hydrocarbons (e.g., sustainable aviation fuel), methanol to gasoline (MTG) or methane to olefin (MTO) products, carbon monoxide via reverse water gas shift (RWGS), or urea or urea ammonium nitrate (UAN). Non-limiting examples of reactor(s) 863 include a Fischer-Tropsch reactor, a high-pressure reactor, an RWGS reactor, a batch reactor, or a continuous flow reactor.

For example, the reactor 863 may be a batch reactor or a continuous flow tubular reactor to produce the synthesis product 864. In one embodiment, the reactor 863 is an RWGS reactor for producing carbon monoxide. In other embodiments, the reactor 863 is a Fischer-Tropsch reactor for producing hydrocarbons, such as alkanes. The reactor 863 may be a bioreactor for producing ethanol or methane. The reactor 863 may be a urea reactor for producing urea. In still other embodiments, the reactor 863 is a tubular reactor, and the synthesis product is methanol. The reactor 863 may be a methanation reactor for producing synthetic natural gas. Other embodiments may include other types of reactors 863 as needed for producing specific synthesis products 864.

Still further, the reactor 863 may also be a general purpose reactor capable of hosting a catalyst, such as a cobalt catalyst, a nickel catalyst, an iron catalyst, or other metal catalysts. In some embodiments, the reactor may be configured to maintain a temperature between 25° C. and 500° C. In some embodiments, the reactor may be configured to maintain a pressure up to 1,000 bar. In some embodiments, the reactor may comprise multiple reaction chambers, which may be fluidly connected, such that each reaction chamber of the reactor may perform a different reaction or multiple instances of the same reaction. In some embodiments, the combined carbon dioxide conversion is at least 60%.

In some embodiments, the hydrogen purification equipment and the reactor are co-located. The carbon dioxide rich stream may be delivered to the reactor without intermediate storage.

The hydrogen purification equipment 860 may also separate nitrogen from the feedstock using equipment such as an additional PSA device to yield a nitrogen-rich gas stream. In some embodiments, the nitrogen-rich gas stream may be directed to an ammonia plant or another offtake facility.

The first purge gas 861 and/or the second purge gas 867 may be directed to an optional power generation plant 806. The first and second purge gases 861, 867 contain unrecovered hydrogen, nitrogen, carbon dioxide, methane, and/or other gases suitable for use by the power generation plant 806 to generate power for the compressors, the purification equipment 860, 866, or third parties such as the grid.

As shown in FIG. 8D, purge gas from the hydrogen purification equipment may be utilized for additional uses as well. In the embodiment of FIG. 8D, the hydrogen production system provides a nitrogen-rich gas stream for downstream use in drilling operations in addition to a hydrogen gas product for use by offtake facilities.

The first-stage or hydrogen purification equipment 860 may include a PSA device or a membrane that provides a purge gas 871 including nitrogen. The nitrogen-containing purge gas 871 may be enriched to increase the nitrogen concentration by receiving additional nitrogen gas 873 from a nitrogen supply system 874, such as a vacuum PSA device or from a power generation plant 806. The enrichment may take place by mixing the purge gas 871 of purification equipment 860 with nitrogen gas 873 from the nitrogen supply system 874 in a mixing manifold (not shown). Further, the nitrogen-rich purge gas 871 may be compressed using a compressor (not shown), with or without the additional nitrogen gas 873, before being directed to a drill rig for use as a drilling fluid 876. For example, the nitrogen-rich purge gas 871 may be compressed to at least 1 MPa, and may have an oxygen molar fraction less than or equal to 1 mol %.

For example, the nitrogen-containing purge gas 871 generated by hydrogen purification equipment 860 and having a nitrogen molar fraction of at least 50 mol % may be used for drilling purposes in various ways. For example, the nitrogen purge gas 871 may be blended with the nitrogen gas effluent 873 of a dedicated air-feed PSA or vacuum PSA to meet the drilling rig demand for an oxygen-depleted drilling fluid, which may have a nitrogen molar fraction of at least 85%. Alternatively, the nitrogen-rich purge gas 871 may be blended with a power purge gas 875 from the power generation plant 806 to yield a low-oxygen, nitrogen-rich stream suitable for drilling fluid. In one or both embodiments, the nitrogen-rich purge gas 871 may have a nitrogen molar fraction of at least 90 mol % after blending.

In still other embodiments, the purge gas 871 may be supplied directly to a rotating-head underbalanced drilling system, or expanded across a turbo-expander for power recovery prior to vent or recycle. In some embodiments, the oxygen content of the drilling fluid may be up to 1 mol %. Because the subsurface hydrogen accumulation is highly reactive with oxygen, use of the nitrogen co-product as the primary drilling fluid substantially reduces the requirement for tube-trailer or liquid-nitrogen deliveries to the drill pad, thereby lowering both operating cost and life-cycle carbon intensity. Further, when the pressure of the nitrogen-rich purge gas 871 is insufficient for downhole circulation, a booster compressor may be employed to increase pressure.

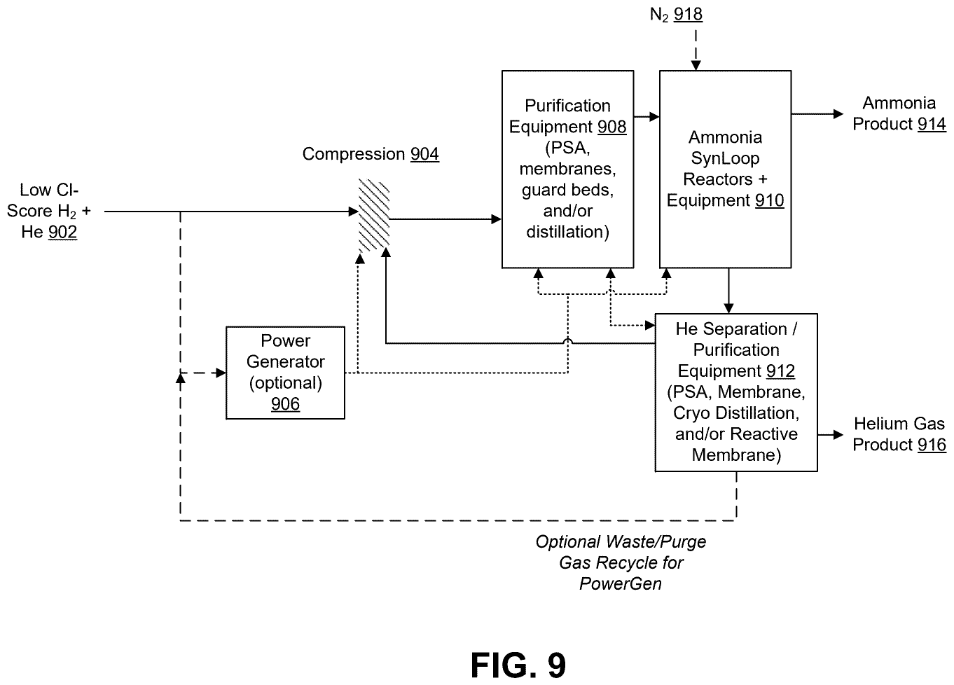

FIG. 9 illustrates an alternative embodiment of a hydrogen production system that includes an ammonia synthesis loop 910 with an intermediate purification equipment 908 that further improves the purity of the hydrogen gas product 902 before entering the ammonia synthesis loop 910. The intermediate purification equipment 908 may include a PSA device, a guard bed, a separation membrane, a reactive membrane, or a cryogenic separation device. The ammonia synthesis loop 910 and the intermediate purification equipment 908 may be fluidically coupled to the first-stage purification equipment 108, 408, 508, 608/610, 710 and/or the second-stage purification equipment 912. By integrating the ammonia synthesis loop 910 within the stages of purification equipment, the input into the ammonia synthesis loop 910 is optimized. The hydrogen production system in FIG. 9 may also receive nitrogen from a separate source 918. Additionally, waste or purge gas from the second-stage purification equipment 912 may be directed to the compressor 904 upstream of the intermediate purification equipment 908, which allows for additional capturing of hydrogen that was unrecovered during the first processing through the ammonia synthesis loop 910.

Further, waste or purge gas from the first-stage purification equipment and the intermediate purification equipment 908 may be directed to the power generation plant 906, and energy from the power generation plant 906 may be directed to the compressor 904, the first stage purification equipment (see FIGS. 1 and 4-8), the intermediate purification equipment 908, the ammonia synthesis loop 910, and the second-stage purification equipment 912. In this way, the hydrogen production system may produce ammonia product 914 and a helium gas product 916, and may do so with lower CI scores than production methods that are not powered in this way.

### Hydrogen Gas Product

In some embodiments, the hydrogen gas product 110 includes hydrogen, helium, and nitrogen. The hydrogen gas product 110 may also include carbon monoxide, carbon dioxide, and methane. In some embodiments, the hydrogen gas product 110 has a purity of greater than 90%. In some embodiments, the hydrogen gas product 110 does not include hydrogen that is produced using electrolysis, steam methane reformation, methane pyrolysis, or gasification.

In one example, the combined hydrogen and helium molar fraction is greater than 98 mol %. In further embodiments, the combined hydrogen and helium molar fraction is greater than 99 mol %, greater than 99.5 mol %, or greater than 99.999 mol %.

In some embodiments, the helium molar fraction of the hydrogen gas product 110 is greater than 0.5 mol %, 1 mol %, 2 mol %, 3 mol %, or 5 mol %.

The hydrogen gas product 110 may have a nitrogen molar fraction of less than 5 mol %, less than 4 mol %, less than 3 mol %, less than 2 mol %, or less than 1 mol %, or less than 0.5 mol %. In other embodiments, the nitrogen concentration may be less than 100 ppm, or less than 30 ppm.

The combined carbon monoxide and carbon dioxide concentration may be less than 50 ppm, or 1 ppm. The carbon monoxide concentration may be less than 0.2 ppm, and the carbon dioxide concentration may be less than 2 ppm.

The methane concentration may be less than 50 ppm, 2 ppm, or 1 ppm. The hydrogen gas product 110 comprises less than 1 ppm sulfur, or 0.4 ppm sulfur.

### Example Analysis

In one example embodiment, the life cycle analysis of the production of a hydrogen gas product 110 from natural hydrogen was explored using a modified version of an open-source oil and gas greenhouse gas calculation tool, the Oil Production Greenhouse Gas Emissions Estimator (OPGEE) v3.0a. Although the OPGEE model is based on conventional oil and gas production, modifications and extensions were made to represent natural hydrogen production. Key changes include modification of gas processing equipment calculations to account for differing composition of the gas, as well as the addition of a simplified pressure-swing absorption (PSA) unit for gas purification.

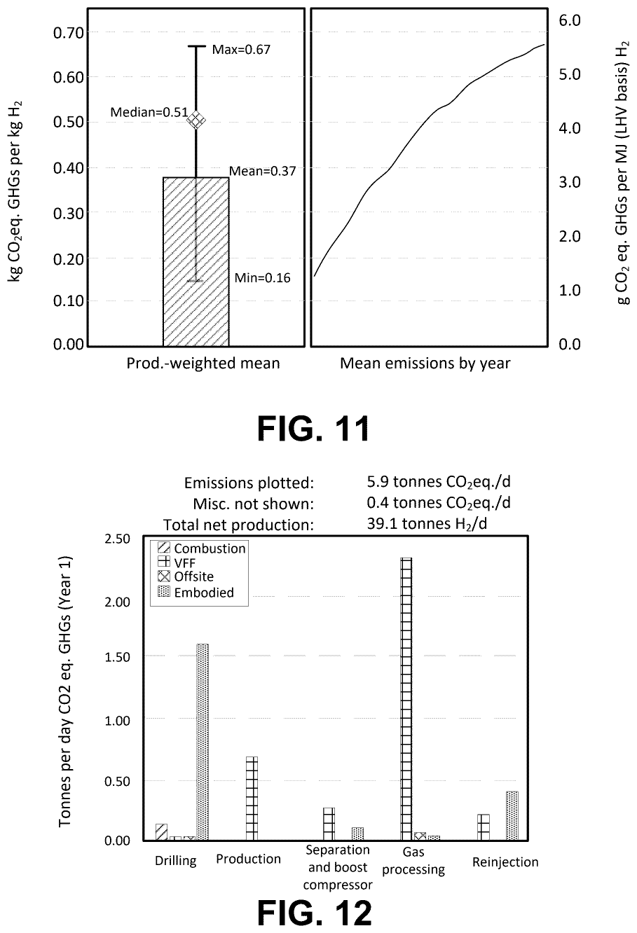

Baseline case results are shown in FIG. 10 and 11. The flow schematic of FIG. 10 for the baseline case shows flows in simulated year 1 of the production process. Self-use of produced clean hydrogen is approximately 8% of gross hydrogen production, while hydrogen lost into the waste gas stream is 10% of gross production. FIG. 11 shows the production-weighted mean emissions for the baseline case in bar form on the left. Production-weighted mean baseline production intensity is 0.37 kg CO₂ eq/kg H₂ produced over the life of the well. The min-max range in CI is given by the year 1 emissions (min) and year 30 emissions (max), as shown in time trend on the right. Emissions increase over time due to reduced well productivity, resulting in more fugitive emissions per unit of gas produced and due to apportioning embodied emissions across fewer units of gas produced. The effect of productivity on fugitive emissions intensity has been seen in multiple empirical methane leakage studies and is well-supported by reasonable models of fugitive emissions causation. Median CI is slightly above the production-weighted mean. FIG. 12 shows a detailed breakdown of emissions sources for year 1 in the baseline case. The emissions are first partitioned into broad stages, such as drilling, production, separation and boost compression, gas processing, and reinjection. Combustion, venting/flaring/fugitive (VFF) emissions, offsite emissions, and embodied emissions are included within each of these broad stages. For clarity, VFF emissions include all purposeful (vented) and unpurposeful (fugitive) emissions from process units and piping. Offsite emissions are emissions that occur offsite in producing goods, services, energy, or other inputs imported to the site. In the baseline case, this category is mostly due to electricity purchases to run remaining electric loads (e.g., dehydration unit solvent circulation pumps), as well as diesel requirements during initial well drilling. In sensitivity cases where the system is electrically driven or uses imported natural gas, then these offsite emissions are larger. Lastly, embodied emissions refer to emissions associated with steel and cement production for those materials consumed during the construction process. These are mostly due to the drilling stage. Wells contribute the majority of embodied emissions because they require large amounts of steel for multiple layers of casing, as well as cement.

The model assumes that waste gas is reinjected into a waste gas disposal well at a similar pressure to the producing well and does not assume that waste gas is reinjected into the productive deposit. Although that kind of reinjection could be done to provide pressure support in the production formation through volumetric replacement of some of the produced gas, it would also result eventually in the breakthrough of large amounts of waste materials like N₂ and CH₄. To avoid this outcome, the model assumes that pressure and production in the reservoir decline in a classic depletion model and that the waste gas is disposed of in a disposal formation.

FIG. 12 does not show the miscellaneous emissions that OPGEE refers to as “small sources.” OPGEE recognizes that not all sources will be tracked in any given model. For example, OPGEE does not track energy use in small trucks driven by workers to and from the job site. This term was added to recognize that there are diminishing returns evident in modeling sources and that there are likely a number of small sources that exist but are not captured by the OPGEE model. In the default case, miscellaneous emissions are 10% of estimated direct sources, excluding embodied and offsite emissions.

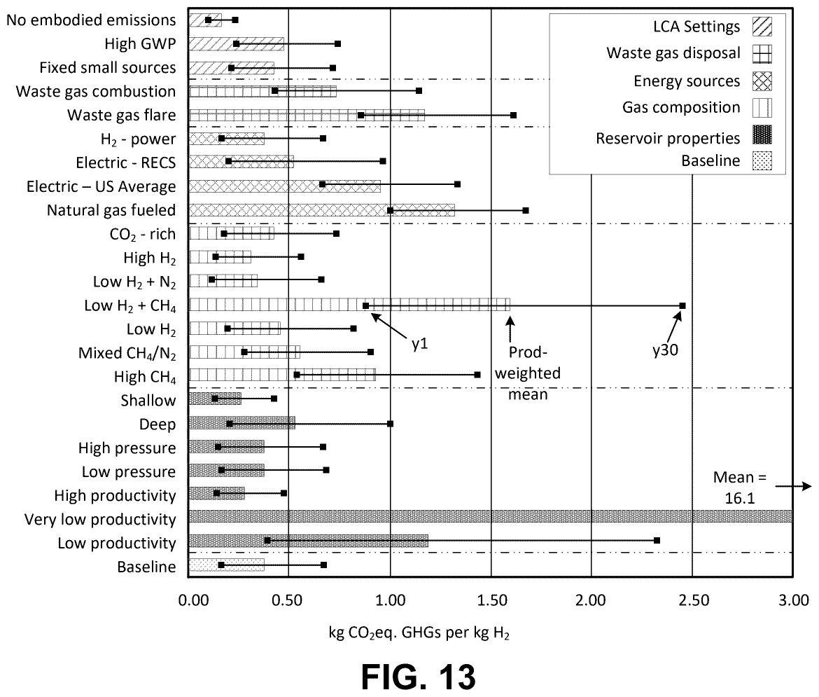

FIG. 13 shows the results of sensitivity analyses for all studied sensitivity cases defined above, where a fixed value of 0.5 g CO₂ equiv/MJ is used. At the bottom are the results for the baseline case. As in FIG. 11 above, the low and high error bars represent year 1 and year 30 emissions intensities as a proxy for variability in emissions from the same project over time. Numerous sensitivity cases have little impact on the CI.

By contrast, some sensitivity cases do end up causing large changes in CI. First, the disposition of the waste gas matters greatly. Although reinjection of waste gas is used in all other cases, dark blue-green bars show cases where (1) the waste gas is used on-site to power systems, then the remainder is reinjected, and (2) a case where waste gas is flared. In the waste gas re-use case, about 30% of the produced waste gas is consumed to power the process, and 70% is reinjected. Because of the carbon content of the waste gas (due to CH₄), this results in higher emissions than the baseline case, though note that net H₂ output as product does increase due to avoiding self-use of the pure H₂ stream, thus reducing the gross-to-net loss of H₂.

Also problematic is the flaring of waste gas, which results in large emissions due to both oxidation of the methane to CO₂ and due to flare slip (OPGEE default values for flare destruction efficiency are used). Importantly, because the waste gas is simply flared, the equipment must still be powered, and therefore net H₂ output to sales is still reduced by the energy requirements of the process. This therefore gives a secondary impact beyond the waste gas consumption case (e.g., the flaring case burns 100% of waste gas rather than 30% and still consumes about 7% of the produced H₂ to power the process). Thus, flaring the associated waste gas is emissions intensive compared with other options. This impact of flaring is even larger in cases where the gas composition is rich in CH₄ rather than N₂ or where the H₂ concentration is low.

Next are cases where the operations are powered with carbon-containing energy sources. A few effects result. First, these cases avoid the parasitic self-use of ˜7%-8% of gross H₂ production, resulting in higher net output. However, they result in higher emissions in both the “Electric—US Average” and “Natural gas” cases. In fact, in the natural gas case, it is clearly strictly better to burn the produced waste gas: this is due to avoiding the upstream emissions associated with imported natural gas and due to the fact that the 10% H₂ slip from the PSA device into the waste gas stream means that per unit heating value, the waste gas from our modeled process has lower CI than US average natural gas from OPGEE.

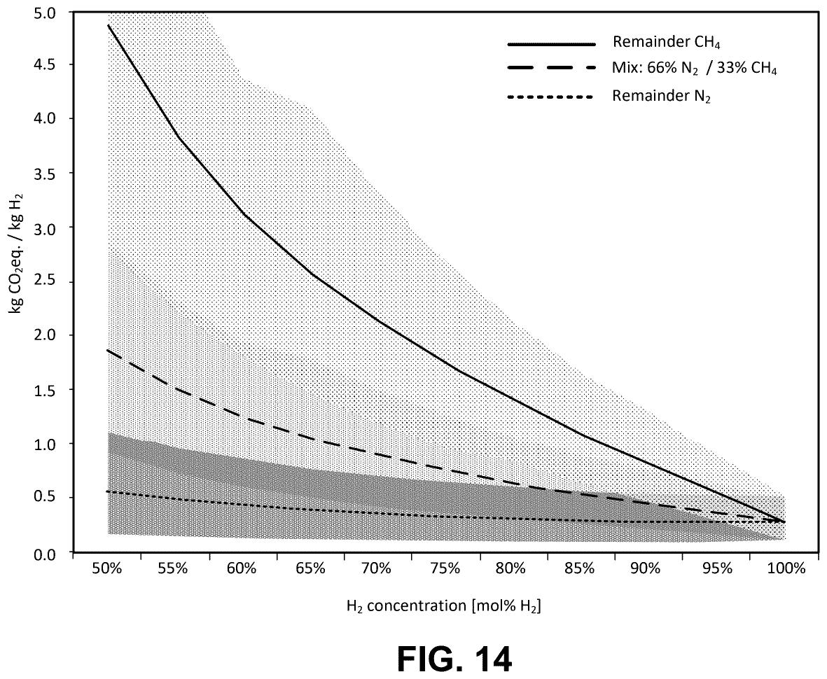

Next are two cases with gas composition high in CH₄ (“High CH₄” and “Low H₂+CH₄”). This increase in CH₄ results in a number of noteworthy impacts. First, all fugitive emissions along the production and processing chain are made more GHG-intensive due to a higher volume fraction of CH₄ in the fugitive emissions stream. Second, the output of useful clean H₂ from the PSA is smaller due to the larger amount of CH₄ in the raw gas stream, rendering all upstream drilling, compression, and dehydration emissions larger per unit of useful clean H₂ produced. Third, the amount of waste gas to reinject gets larger, necessitating increased parasitic self-use of produced H₂ and further reducing net H₂ outputs to the consumer. In cases below 75 mol % H₂, this effect gets increasingly large as the concentration drops due to these interacting impacts (see FIG. 14).

FIG. 14 sweeps over a wide range of concentrations. Although lower H₂ concentrations are certainly found in the literature on gas sample collection, actual economic developers interested in natural H₂ as a sustainable energy source would tend to focus on resources with higher H₂ concentrations. Because developers will tend to focus on higher grade resources, it is unlikely that accumulations with low H₂ concentration would be economic to develop.

The last major driver of emissions is treatment of embodied emissions. The importance of these is illustrated by the low emissions in the “no embodied emissions” case, which has a CI well below that of our baseline case. These impacts are also illustrated by the higher emissions intensity of the low-productivity case, wherein cumulative production per well drops to 25% of the baseline value (0.33 billion standard cubic feet (BCF) per well as compared with 1.33 BCF/well), and therefore embodied steel and cement emissions must be apportioned over a smaller amount of energy produced. In some life cycle models, such as the GREET model from Argonne National Laboratory, embodied emissions from various H₂ production processes are not included by default, which would result in figures systematically lower than those produced by OPGEE.

### Discussion of Example Analysis

This prospective analysis of a generic natural H₂ production process shows that it is possible to extract low-CI H₂ under reasonable assumptions about gas compositions, pressures, and production practices. If a clean energy source is used to power production processes (e.g., self-produced H₂ or clean power), most of the remaining emissions are due to fugitive emissions of the raw gas during production and processing and embodied emissions. In our baseline case, loss of H₂ as fugitive emissions to the transport inlet is modeled to be 0.8% of total wellhead H₂ production (year 1 value). Although in line with other values in the literature (e.g., GREET model assumes 0.62%), diligent operators could possibly outperform this value. In addition to the significant uncertainty regarding fugitive emissions from oil and gas systems in general, it is still unclear how closely the fugitive emissions rates in OPGEE—derived from field studies of oil and gas production—will estimate those emissions for H₂ production and processing.

Another possibly important factor is to consider the counter-factual case for what would have happened to the H₂ in a natural system without extraction. It is possible that long-term interaction between H₂ production and natural H₂ seeps could exist, such that production and consumption of H₂ from the subsurface could result in less natural seepage over time. This would potentially offset leakage impacts from H₂ use by avoiding natural seepage. Given the large estimates of natural flux from the earth of 0.1-10 Tg H₂/year noted above, atmospheric impacts should account for how much perturbation is provided by any human activity.

It is worthwhile to compare our results to others from the literature for other sources of H₂, such as renewable-powered electrolysis or steam methane reforming with CO₂ capture. As noted above, the GREET model does not include embodied energy in emissions, so it gives wind- and solar-derived H₂ a CI of 0 kg CO₂/kg H₂. Those results are not comparable to the data here, which do include embodied emissions. Numerous other studies of green and blue H₂ have been performed that do include embodied emissions. First, NETL performed detailed analyses of steam methane reforming systems, finding 16.4 kg CO₂ equiv/kg H₂ for the case without carbon capture and storage (CCS) and 8.9 kg CO₂ equiv/kg H₂ for the case with CCS. Kanz et al. found an average CI for solar-photovoltaic (PV)-derived H₂ of 3.6 kg CO₂/kg H₂, with a 95% CI on the empirical data of 1.1-6.4 kg CO₂ equiv/kg H₂ (min-max=0.7-6.6). Numerous other studies exist, but there is little consensus in the literature, with Kanz et al. noting that only 14 of 33 studies could be harmonized due to lack of open data and stating that “Due to the lack of transparency of most LCAs included in this review, full identification of the sources of discrepancies (methods applied, assumed production conditions) is not possible.” Despite this uncertainty, the values estimated here for natural H₂ are lower than those of green and blue H₂ for most near-term modeling cases (that include embodied emissions).

Importantly, the global warming potential (GWP) of H₂ is a matter of some recent debate, with 100-year GWPs in recent studies varying by a factor of 3 or so from the lowest to highest estimates. Shifting from the baseline GWP₁₀₀ of 5 to a high estimate of 11 does not result in major changes to the GHG intensity (see FIG. 13). Shifting to a GWP₂₀ instead of the OPGEE default of GWP₁₀₀ would have a larger impact, but in that case, other sources such as natural gas would also have higher GHG intensity.

A key factor in low-CI production is the source of energy to power and heat for compression and gas processing. These results suggest that H₂ could be a low-carbon source of energy if the gas is produced responsibly and the system is powered by low-carbon energy sources, either by self-produced clean H₂ or by purchased certified green power (e.g., “Electric—RECS” case above, where RECS represents renewable energy certificates representing green attributes of power that can be purchased to offset emissions).

The baseline case has a production-weighted average CI for extraction and processing of ˜3 g CO₂ equiv/MJ, or ˜20%-33% the production CI of conventional natural gas sources. Additionally, the end-product H₂ has no combustion CI, compared with ˜50 g CO₂ equiv/MJ for natural gas. Thus, the overall life cycle CI for natural H₂ (including transport and end-use of the gas—though not modeled explicitly here) is likely to exhibit a 90%-95% reduction compared with conventional gas.

Importantly, these results can be placed in the context of H₂ production incentives. For example, the US Inflation Reduction Act (IRA) incentivizes H₂ differently depending on its CI. The most stringent tranche requires a CI of 0.45 kg CO₂ equiv per kg of H₂ produced, while the decreasing incentives are available up to 4 kg CO₂ equiv per kg H₂ produced. The production cases modeled here generally fall into the most stringent or second-most stringent CI tranche. The only pathways modeled here that fail to align with these targets are the most CH₄-rich cases shown in FIG. 14, which have significant fugitive emissions impacts and larger separation and reinjection losses. These results give a first indication that natural H₂ may be competitive for clean H₂ production standards.

### Conclusion

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A method of producing a hydrogen gas product and a carbon dioxide-rich gas stream, the method comprising:
receiving feedstock from a geologic hydrogen source;
separating, through a first pressure swing adsorption (PSA) device in a hydrogen purification equipment, carbon dioxide from hydrogen in the feedstock to provide an effluent including hydrogen and a first purge gas including carbon dioxide;
separating, through carbon dioxide purification equipment, carbon dioxide from other gases in the first purge gas to provide a carbon dioxide-rich gas stream having a carbon dioxide molar fraction of at least 50 mol % and a second purge gas; and
reacting, in a reactor, the carbon dioxide-rich gas stream with hydrogen of the effluent to produce a synthesis product, wherein the synthesis product comprises one or more of synthetic natural gas, carbon monoxide, methanol, ethanol, Fischer-Tropsch hydrocarbons, olefins, urea, ammonia, or urea ammonium nitrate.

2. The method of claim 1, wherein the reacting step comprises:
reacting the carbon dioxide-rich stream and the hydrogen in a reverse water gas shift reactor to produce carbon monoxide; and
reacting the carbon monoxide with hydrogen in a Fischer-Tropsch reactor.

3. (canceled)

4. The method of claim 1, wherein the reactor is a batch reactor or a continuous flow reactor.

5. The method of claim 1, wherein the reactor is a bioreactor, and the synthesis product comprises ethanol or methane.

6. The method of claim 1, wherein the reactor is a reverse water gas shift reactor, and wherein the synthesis product comprises carbon monoxide.

7. The method of claim 1, wherein the reactor is a Fischer-Tropsch reactor, and wherein the synthesis product comprises hydrocarbons.

8. The method of claim 1, wherein the reactor is a urea reactor, and wherein the synthesis product is urea.

9. The method of claim 1, wherein the reactor is a tubular reactor, and wherein the synthesis product is methanol.

10. The method of claim 1, wherein the reactor is a methanation reactor, and wherein the synthesis product is synthetic natural gas.

11. The method of claim 1, further comprising:
separating, through a third PSA device in the hydrogen purification equipment, nitrogen from hydrogen in the feedstock or the effluent from the first PSA device to provide a nitrogen-rich gas stream; and
directing the nitrogen-rich gas stream to an ammonia plant.

12. (canceled)

13. The method of claim 1, wherein the hydrogen purification equipment is configured to receive the feedstock and produce the hydrogen gas product, and wherein production of the hydrogen gas product exhibits a carbon intensity score less than 4.0 kg CO₂ eq/kg H₂.

14-17. (canceled)

18. A system for producing a hydrogen gas product and a carbon dioxide-rich gas stream, the system comprising:
hydrogen purification equipment configured to receive feedstock from a geologic hydrogen source, wherein the hydrogen purification equipment is configured for separating carbon dioxide from hydrogen in the feedstock to provide an effluent including hydrogen and a first purge gas including carbon dioxide, wherein the hydrogen purification equipment includes a first pressure swing adsorption (PSA) device;
carbon dioxide purification equipment for separating carbon dioxide from other gases in the first purge gas to provide the carbon dioxide-rich gas stream having a carbon dioxide molar fraction of at least 50 mol % and a second purge gas, wherein the carbon dioxide purification equipment includes a secondary PSA device, a temperature-swing adsorption, a separation membrane, a chemical-solvent absorption, or a cryogenic distillation; and
a reactor configured to receive hydrogen of the effluent and the carbon dioxide-rich gas stream and to produce a synthesis product, wherein the synthesis product comprises one or more of synthetic natural gas, carbon monoxide, methanol, ethanol, Fischer-Tropsch hydrocarbons, olefins, urea, and urea ammonium nitrate.

19. The system of claim 18, further comprising an offtake facility including the reactor, and wherein the offtake facility includes a reverse water gas shift reactor and a Fischer-Tropsch reactor, and wherein the offtake facility is configured to produce the synthesis product with a combined carbon dioxide conversion of at least 60%.

20. (canceled)

21. The system of claim 18, wherein the reactor is a batch reactor or a continuous flow reactor.

22. The system of claim 18, wherein the reactor is a bioreactor, and the synthesis product comprises ethanol or methane.

23. The system of claim 18, wherein the reactor is a reverse water gas shift reactor and the synthesis product comprises carbon monoxide.

24. (canceled)

25. The system of claim 18, wherein the reactor is a Fischer-Tropsch reactor, and wherein the synthesis product comprises alkanes.

26. The system of claim 18, wherein the reactor is a urea reactor and the synthesis product is urea, wherein the reactor is a tubular reactor and the synthesis product is methanol, or wherein the reactor is a methanation reactor and the synthesis product is synthetic natural gas.

27-30. (canceled)

31. The system of claim 18, wherein the hydrogen purification equipment is configured to receive the feedstock and produce the hydrogen gas product, and wherein production of the hydrogen gas product exhibits a carbon intensity score less than 4.0 kg CO₂ eq/kg H₂.

32-139. (canceled)
